(19) United States
(12) Patent Application Publication
(10) Pub. No.: US 2023/0255684 A1
(43) Pub. Date: Aug. 17, 2023

(54) EGM FREQUENCY ANALYSIS FOR LESION EVALUATION

(71) Applicant: Medtronic, Inc., Minneapolis, MN (US)

(72) Inventors: Megan M. Schmidt, Blaine, MN (US); Daniel C. Sigg, St. Paul, MN (US); Lars M. Mattison, Blaine, MN (US); Tomaz Jarm, Ljubljana (SI); Jernej Stublar, Ljubljana (SI); Damijan Miklavcic, Kranj (SI); Nicolas Coulombe, Anjou, CA (US)

(21) Appl. No.: 18/160,277

(22) Filed: Jan. 26, 2023

Related U.S. Application Data
(60) Provisional application No. 63/267,868, filed on Feb. 11, 2022.

(51) Int. Cl. A61B 18/14

(52) U.S. Cl. CPC A61B 18/1492; A61B 2018/00577

(57) ABSTRACT

Evaluating a cardiac lesion formed by an ablation procedure, by receiving, by processing circuitry and following conclusion of delivery of ablation energy, a bioelectrical signal from an electrode proximate to a target location of cardiac tissue for the cardiac lesion; determining, by the processing circuitry, one or more characteristics of the received bioelectrical signal in a frequency band of the received bioelectrical signal; and estimating, by the processing circuitry, an efficacy of the cardiac lesion based on a comparison of the determined amplitude of the bioelectrical signal and a threshold amplitude.

This application claims the benefit of U.S. Provisional Pat. Application 63/267,868, filed 11 Feb. 2022, the entire content of which is incorporated herein by reference.

### TECHNICAL FIELD

The disclosure relates to ablation of cardiac tissue, more specifically, to techniques to determine the efficacy of a cardiac lesion formed by ablation.

### BACKGROUND

Cardiac ablation is a procedure that may be employed to treat an irregular heart rhythm (e.g., an arrhythmia). Cardiac ablation may involve damaging (e.g., scarring) heart tissue to disrupt generation and/or propagation of faulty electrical signals causing the arrhythmia. Ablation devices may include catheters with one or more electrodes. The electrodes may be configured to direct ablation energy to tissue of a patient to cause a lesion in the tissue, for example to block unwanted bioelectrical signal conduction.

### SUMMARY

In general, the disclosure describes techniques to evaluate specified single or multiple frequency bands of measured intracardiac electrogram (iEGM) signals to evaluate lesion formation caused by ablation. A practitioner, e.g., an electrophysiologist may evaluate a lesion to determine whether the lesion will perform as intended, e.g., will sufficiently disrupt or block unwanted electrical signals. Different frequency ranges of recorded signals may provide information for different ablation techniques. For example, for pulsed field ablation (PFA), the iEGM may show a reduction in amplitude in a relative high frequency range of the iEGM signal, such as 63 Hz - 500 Hz frequencies immediately after ablation, e.g., between zero and five minutes post ablation. In contrast, increase in amplitude in a low frequency range, such as 0 Hz - 8 Hz may be present after PFA. Allowing the tissue to recover, e.g., for more than five minutes, may show iEGM amplitude reduction in this lower frequency range, which may correlate to chronic lesion formation.

Similarly, for radio frequency ablation (RFA), iEGM amplitude reduction may be most prevalent in the 125 Hz - 250 Hz range between zero and five minutes post ablation, while iEGM amplitude reduction may be visible in the 0 Hz - 8 Hz range after the tissue recovers, for example between five and 30 minutes post ablation. In this manner, the techniques of this disclosure may evaluate the lesion formation as well as the likelihood that the lesion will be durable and continue to chronically block bioelectrical signals.

Therefore, this disclosure presents techniques to analyze the iEGM signal in the time and frequency domain to predict chronic lesion size and/or its chronic persistence after ablation. This disclosure may provide analysis techniques to develop an index, e.g., a lesion durability index, to assess lesion formation based on iEGM characteristics, for example, by selecting and analyzing iEGM components from one or more frequency bands. In some examples the index may also include other biological measurements such as temperature, impedance, and similar measurements. The specific analysis details, e.g., number of frequency bands, bandwidth of each frequency band, total frequency range of recorded signals, the timing of when to measure the iEGM and so on may differ for different types of ablation. To simplify the description, this disclosure may present examples that focus on iEGM signal analysis for PFA, but the techniques in general may apply to other types of ablation, e.g., RFA, cryo-ablation, and so on.

In one example, the disclosure describes a method for evaluating a cardiac lesion formed by an ablation procedure, the method comprising: receiving, by processing circuitry and following conclusion of delivery of ablation energy, a bioelectrical signal from an electrode proximate to a target location of cardiac tissue for the cardiac lesion; determining, by the processing circuitry, one or more characteristics, such as an amplitude of the received bioelectrical signal in a frequency band; and estimating, by the processing circuitry, an efficacy of the cardiac lesion based on a comparison of the determined amplitude of the bioelectrical signal and a threshold amplitude. In other examples, the disclosure describes an ablation device and a medical system configured to evaluate a cardiac lesion as described in the method above.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a block diagram illustrating an example system to deliver ablation energy as well as to measure bioelectrical signals.

FIGS. 2A and 2B are conceptual diagrams illustrating examples of ablation catheters configured to both deliver ablation energy and measure bioelectrical signals.

FIG. 3 includes time graphs illustrating example of iEGM measurements before, immediately after and 5 minutes after ablation for both unipolar and bipolar configurations.

FIG. 4 includes time charts illustrating examples of discrete wavelet transformation decomposition of measured iEGM before and after ablation for a bipolar configuration with a clinical bandwidth of 30-500 Hz.

FIG. 5 includes time charts illustrating examples of discrete wavelet transformation decomposition of measured iEGM before and after ablation for a unipolar configuration (bandwidth 0.5-500 Hz).

FIG. 6A is a chart illustrating the results of ablation on measured bipolar relative peak-to-peak values over the wide clinical bandwidth.

FIG. 6B is a chart illustrating the results of ablation on unipolar measured relative peak-to-peak values for the high frequency (63 Hz - 500 Hz) bands.

FIGS. 7A and 7B are charts illustrating an amount of correlation between measured lesion volume (assessed from gross pathology 6-7 weeks post-ablation) and measured bipolar peak-to-peak values at 30 seconds post ablation and 3.5 minutes post ablation respectively over the wide clinical bandwidth.

FIGS. 7C and 7D are charts illustrating an amount of correlation between measured lesion volume (assessed from gross pathology 6-7 weeks post-ablation) and measured unipolar peak-to-peak values at 30 seconds post ablation and 3.5 minutes post ablation respectively for the high frequency (63 Hz - 500 Hz) bands.

FIGS. 8A and 8B are time charts illustrating a post-ablation ST elevation-like phenomenon as measured in time domain and in the lower frequency band (0 Hz - 8 Hz).

FIG. 9 is a chart illustrating the results of ablation on unipolar measured relative peak-to-peak values for the low frequency (0 Hz - 8 Hz) band (p<0.05, ANOVA and repeated measures ANOVA with post hoc tests used for “between doses: * *” and “within dose: *” comparisons).

FIGS. 10A and 10B are charts illustrating an amount of correlation between measured lesion volume (assessed from gross pathology 6-7 weeks post-ablation) and measured unipolar peak-to-peak values for the low frequency (0 Hz - 8 Hz) band at 30 seconds and 3.5 minutes post ablation.

FIG. 11 is a flowchart illustrating an example operation of the lesion analysis system of this disclosure.

FIGS. 12A and 12B are time charts illustrating a post-ablation area under the curve (AUC) analysis as measured in time domain for the ST segment of the cardiac cycle.

FIGS. 13A and 13B are charts illustrating the correlation of the parameter derived from AUC analysis (average ST segment amplitude) to measured lesion volume based on measurements taken 30 seconds after ablation as well as 3.5 minutes after ablation.

FIG. 14 is an example implementation of a grid catheter which may be used for measuring cardiac signals according to one or more techniques of this disclosure.

FIGS. 15A and 15B are graphs illustrating the impact of applying ablation to different regions i.e. to necrotic tissue (scar), to healthy tissue (healthy) and to heterogeneous tissue near necrotic tissue (border), e.g., for lesion homogenization.

FIGS. 16A and 16B are graphs illustrating the impact of applying ablation in healthy atrial tissue, e.g., for pulmonary vein isolation. The results are presented separately for PFA and RFA ablation modalities and separately for transmural (T+) and non-transmural (T-) lesions (based on gross pathology examination of the hearts).

### DETAILED DESCRIPTION

In some cases, it may be desirable for a practitioner to evaluate a lesion created by ablation. For instance, the practitioner may evaluate the lesion to determine whether the lesion will perform as intended (e.g., will sufficiently disrupt abnormal electrical signals to treat the underlying condition such as an arrhythmia). To evaluate the lesion, the practitioner may place electrodes on or near cardiac tissue in which the lesion is formed or is forming to measure bioelectrical signals, such as intracardiac electrograms (iEGM). The measured bioelectrical signals may indicate one or more aspects of the lesion. For instance, a peak-to-peak amplitude of an iEGM signal may provide an indication of whether the application of ablation energy formed a lesion that will perform as intended. Specifically, when evaluating a lesion created using cryoablation or radio frequency ablations (RFA), a reduction in iEGM amplitude may indicate acute lesion formation. Pulsed electrical fields, e.g., irreversible electroporation, may also be used for lesion formation. Electroporation is a physical method that uses short high-intensity electrical pulses to permeabilize cell membranes with numerous possible consequences for the cells. For example, pulsed field ablation (PFA)), while intended to ablate or kill the myocardial cells via mechanisms of irreversible electroporation, may also cause acute stunning of myocardial cells via transient membrane permeabilization and membrane channel incapacitation and result in an acute amplitude reduction in a measured iEGM.

Electrodes either for ablation energy delivery, or to measure iEGMs may be configured as bipolar or unipolar. A bipolar configuration is one in which one or more positive electrodes and negative electrodes are placed near the tissue to be measured, or to be ablated. Electrodes may also be configured as unipolar, in which, for example, a first electrode or electrodes may be in contact with the tissue to be measured, or ablated, and one or more return electrodes may be placed some distance from the first electrodes, e.g., a few centimeters distance away, such as a pad electrode on the skin of the patient. Additional details on bipolar and unipolar measurements are listed in the table below:

| | | |
|---|---|---|
| Parameter | Unipolar iEGM | Bipolar iEGM |
| Field of view | Wide (Far-Field) | Narrow (Near-Field) |
| Signal to noise ratio | Small | Big |
| Sensitive to wavefront direction | NO | YES |
| Signal amplitude depends on electrode size | YES | YES |
| Signal amplitude depends on electrode spacing | NO | YES |
| Clinical frequency bandwidth recording settings | Ex. 1: 2 Hz - 240 Hz | Ex. 1: 16 - 500 Hz |
| | Ex. 2: 2 Hz - 100 Hz | Ex. 2: 30 - 300 Hz |

This disclosure describes techniques to evaluate specified multiple frequency bands of measured bioelectrical signals, including intracardiac electrogram (iEGM) signals as well as other types of bioelectrical signals, to evaluate lesion formation. To simplify the explanation, the disclosure may focus on iEGM as an example of bioelectrical signals. However, the techniques of this disclosure may also apply to other types of signals, such as cardiac signals received via epicardial electrodes, intravenous electrodes, such as via the coronary sinus with access to cardiac veins such as the vein of Marshall, electrodes placed in the esophagus or other locations. In some examples, processing circuitry of this disclosure may analyze the iEGM signal, or other signals, in the time and frequency domain to predict chronic lesion efficacy after ablation, where lesion efficacy describes the ability of the lesion to chronically prevent undesirable cardiac signals from originating in or propagating through the ablated tissue. Lesion efficacy may depend on one or more lesion characteristics including lesion depth, transmurality, surface area and volume. Said another way, lesion efficacy indicates that the lesion may substantially block myocardial propagation between adjacent tissue segments chronically (permanently in an ideal case). Transmural means that the extent of tissue damage is such that the substrate or tissue damaged by the ablation procedure to create the lesion covers the surface of the lesion site and extends through the thickness of the tissue wall. The transmurality of the lesion may also indicate when the necrosis is sufficient to extend from the endocardial to the epicardial layer.

This disclosure may provide analysis techniques to develop an index to assess lesion formation based on iEGM characteristics, for example, by analyzing specific frequency spectra of measured iEGMs. In some examples the index, e.g., a lesion durability index, may also include other measurements such as temperature, monophasic action potential (MAP) waveform properties, impedance, and so on. The specific analysis details of the lesion characterization process, e.g., number of frequency bands, bandwidth of each frequency band, frequency range, the timing of when to measure the iEGM and so on may differ for different types of ablation.

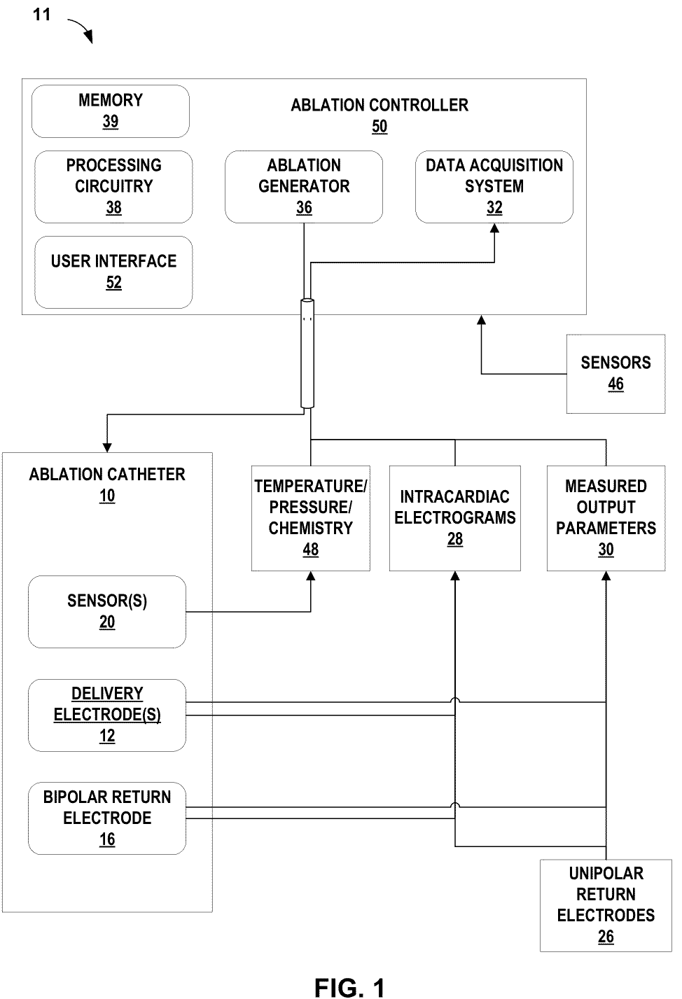

FIG. 1 is a block diagram illustrating an example system to deliver ablation energy as well as to measure bioelectrical signals. The example of FIG. 1 illustrates a medical system for performing an ablation procedure, which may include an ablation catheter 10 in an ablation system 11. The medical system of FIG. 1 may also include one or more other sensors 46, which may provide data to processing circuitry 38, and/or to other processing circuitry of the medical system (not shown in FIG. 1). In some examples, medical system 11 may include an ablation device such as ablation catheter 10. In other examples, the ablation device may include one or more additional components depicted in FIG. 1 such as ablation generator 36, user interface 52, processing circuitry 38 or any other combination of components depicted in FIG. 1. In other examples, medical system 11, and an associated ablation device, may include more of fewer components than depicted by FIG. 1.

The ablation catheter 10 is configured to perform electrical measurements and temperature measurements of tissue that is to be ablated or has been ablated. In some examples, ablation catheter 10 may be configured to perform other types of measurements including pressure, chemistry (e.g. nicotinamide adenine dinucleotide (NAD) or reduced NAD (NADH), impedance and so on. One or more ablation delivery electrodes 12, bipolar return electrode 16 and unipolar return electrode 26 may provide electrical measurements of bioelectrical signals. In some examples, thermocouples and other types of sensors that may be included with sensors 20 may provide the temperature, and other measurements. In some examples, sensors 46, which may be separate from ablation catheter 10 may also perform one or more measurements such as thoracic impedance, cardiac rhythm, a blood chemistry measurement, echocardiogram, ultrasound imagery and so on.

In the example of FIG. 1, ablation controller 50 includes processing circuitry 38 operatively coupled to memory 39, as well as to user interface 52, ablation generator 36 and data acquisition system 32. Ablation controller may receive settings and configuration instructions for ablation delivery and for data acquisition via user interface 52, which may include switches, knobs, one or more displays, including a touch screen display, indicator lights, text, and numerical entry keys and so on. Processing circuitry 38 may control the operation of ablation controller 50 based on programming instructions store, for example, at memory 39 and may receive bioelectrical signals, e.g., via data acquisition system 32, sensors 46 and so on.

Data acquisition system 32 may receive ablation parameters 30 that may include one or more of power, duration, pulse width, voltage, frequency, number of pulses, etc., and/or other parameters from an ablation generator 36. These parameters may be input by the operator of the ablation system 11, e.g., via user interface 52. For example, the ablation generator 36 may be circuitry configured to provide RF energy, delivered to the cardiac tissue to cause ablation. In other examples ablation system 11 may be configured to deliver pulsed field ablation (PFA) e.g., PEF (pulsed electric field) ablation.

The delivery of the ablation energy may be by direct electrical contact and/or by electromagnetic transfer. In other examples, the ablation generator 36 may provide laser ablation. In some examples, the ablation generator 36 may provide cryoablation. Any one or more ablation energy sources may be employed, of the same type or of different types. The ablation parameters 30 may be input by an input device of user interface 52 (such as a keyboard, mouse, etc.) of the data acquisition system 32, the data acquisition system 32 transmits such inputs to the ablation generator 36. The ablation generator 36 may output pulsed electric fields or RF energy, for example, to the catheter 10 to be applied to the cardiac tissue to form the lesion.

Examples of processing circuitry 38 in ablation controller 50 may include any one or more of a microcontroller (MCU), e.g. a computer on a single integrated circuit containing a processor core, memory, and programmable input/output peripherals, a microprocessor (µP), e.g. a central processing unit (CPU) on a single integrated circuit (IC), a controller, a digital signal processor (DSP), an application specific integrated circuit (ASIC), a field-programmable gate array (FPGA), a system on chip (SoC) or equivalent discrete or integrated logic circuitry. A processor may be integrated circuitry, i.e., integrated processing circuitry, and that the integrated processing circuitry may be realized as fixed hardware processing circuitry, programmable processing circuitry and/or a combination of both fixed and programmable processing circuitry. Accordingly, the terms “processing circuitry,” “processor” or “controller,” as used herein, may refer to any one or more of the foregoing structures or any other structure operable to perform techniques described herein. Processing circuitry 38 in the example of FIG. 1 may include other processing circuitry in system 11. For example, data acquisition system 32 may also include processing circuitry.

Examples of memory 39 may include any type of computer-readable storage media include random access memory (RAM), read only memory (ROM), programmable read only memory (PROM), erasable programmable read only memory (EPROM), one-time programable (OTP) memory, electronically erasable programmable read only memory (EEPROM), flash memory, or another type of volatile or non-volatile memory device. In some examples the computer readable storage media may store instructions that cause the processing circuitry to execute the functions described herein. In some examples, the computer readable storage media may store data, such as configuration information, temporary values and other types of data used to perform the functions of this disclosure.

Ablation generator 36 may include a pulsed field ablation (PFA) generator configured to deliver, for example, pulsed electric field energy to a tissue area in proximity to the treatment region(s). Ablation catheter 10 may be a medical device passable through a patient’s vasculature and positionable proximate to a tissue region for diagnosis or treatment. Ablation catheter 10 may include one or more treatment region(s) configured to monitor, diagnose, and/or treat a portion of a patient. The treatment region(s) may have a variety of configurations to facilitate such operation. In the case of bipolar pulsed field delivery, catheter 10 may include delivery electrode 12 and bipolar return electrode 16 that form the bipolar configuration for energy delivery where energy passes between one or more electrodes and one or more different electrodes on the same electrode array. In some examples, delivery electrodes 12, bipolar return electrodes 16 and unipolar return electrodes 26 may each comprise a plurality of the electrodes.

The unipolar electrodes 26 may comprise one or more surface ECG electrodes on the patient in communication with ablation generator 36. In some examples, one or more of the electrodes of system 11 may monitor the patient’s cardiac activity for use in determining pulse train delivery timing at the desired portion of the cardiac cycle, for example, during the ventricular refractory period. In addition to monitoring, recording or otherwise conveying measurements or conditions the electrodes of system 11 may provide to processing circuitry 38, temperature, electrode-tissue interface impedance, measured output parameters 30, such as delivered charge, current, power, voltage, energy, or the like.

System 11 may be configured to deliver and evaluate a cardiac lesion formed by an ablation procedure. A practitioner may insert ablation catheter 10 into the atrium of a patient, e.g., for patients with atrial fibrillation, or the ventricle of a patient. Processing circuitry 38 may receive one or more bioelectrical signals from an electrode or other sensors proximate to a target location of cardiac tissue for the cardiac lesion. The practitioner may move the catheter as needed to determine where to locate a lesion to block or attenuate unwanted cardiac activity and/or conduction. In other words, the practitioner may determine a position of delivery electrodes 12 and/or bipolar return electrodes 16 relative to the target location based on the received bioelectrical signals, or other signals.

Following conclusion of delivery of ablation energy, processing circuitry 38 of medical system 11 may receive a bioelectrical signal from an electrode proximate to a target location of cardiac tissue for the cardiac lesion. As noted above, in some examples delivery electrode 12 is a single electrode, while in other examples, delivery electrodes 12 is one of a plurality of electrodes. For receiving a bipolar bioelectrical signal, at least two electrodes of the plurality of electrodes are in contact with or proximate to the cardiac tissue. Ablation catheter 10 may have several electrodes, any of which may be configured as a delivery electrode 12 and any of which may be configured as a bipolar return electrode 16. The system of this disclosure may deliver ablation energy in either a bipolar configuration or a unipolar configuration. The system of this disclosure may also receive bioelectrical signals, such as iEGM in either a bipolar configuration or a unipolar configuration. In some examples, the system may use the same electrodes for both delivery of ablation energy and for receiving signals. In other examples, the system may deliver ablation energy through a first electrode configuration and receive bioelectrical signals with a different electrode configuration.

In the example of a unipolar bioelectrical signal, one or more delivery electrodes 12 may be in contact with or proximate to the cardiac tissue, while a second electrode of the plurality of electrodes, e.g., one or more unipolar return electrodes 26 is separate from delivery electrode 12. In some examples unipolar return electrodes 26 include one or more patch electrodes which may be placed on the patient’s skin. In other examples, unipolar return electrodes 26 may include an intra-ventricular electrode, such as for an atrial ablation, an electrode in contact with the pericardium, additional implanted electrode, or electrodes at some other location on the patient, such as in the inferior vena cava. In other words, in a unipolar configuration the return/reference electrode 26 may be any electrode, or electrodes, not in contact with cardiac tissue.

Once positioned, processing circuitry 38 may receive and store pre-ablation bioelectrical signals at memory 39, such as intracardiac electrograms 28, as well as other sensed signals 48 via sensors 20 or via sensors 46. Pre-ablation signals may also be referred to as baseline bioelectrical signals in this disclosure. System 11 may deliver ablation energy to the target location, e.g., generated by ablation generator 36 and via the electrodes on ablation catheter 10, or a combination of delivery electrode or electrodes 12 and unipolar return electrodes 26. In some examples, the ablation energy is in the form of PFA.

Processing circuitry 38 may receive a second bioelectrical signal, e.g., via data acquisition system 32 and electrodes on ablation catheter 10 and/or unipolar return electrode 26 during or shortly after delivery of the ablation energy. In this disclosure, ‘during or shortly after’ means as soon as the circuitry and cardiac tissue has recovered enough from the delivered ablation energy to provide bioelectrical signals that the sensors or various electrodes may sense.

Processing circuitry 38 may determine one or more characteristics of the received bioelectrical signal in one or more frequency bands. Some examples of characteristics may include an amplitude, such as a peak-to-peak voltage, other voltage differences between characteristic fiducial points, signal slopes, power of parts of the signal in time or frequency domain or other parameters reflecting measurable changes in iEGM morphology as a result of PFA. To simplify the description, this disclosure may focus on measuring amplitude of the bioelectrical signal. However, processing circuitry 38 may use any measurable characteristics of received signals to determine lesion efficacy. The processing circuitry may estimate an efficacy of the cardiac lesion based on a comparison of the determined amplitude of the bioelectrical signal and a threshold amplitude. In some examples, the processing circuitry may determine the threshold amplitude based on the baseline bioelectrical signal received before the delivery of the ablation energy. In other words, the techniques of this disclosure may evaluate whether the iEGM assessment characteristics are associated with irreversible electroporation and ultimately a durable as well as transmural lesion that may relieve, or at least improve, negative symptoms for a patient.

In some examples, processing circuitry 38 may perform a frequency domain analysis on the received, e.g., pre-ablation or post-ablation, bioelectrical signal. The frequency analysis may include dividing the received second bioelectrical signal into two or more frequency bands. Processing circuitry 38 may perform any type of frequency domain analysis, including Fourier analysis, e.g., fast Fourier transforms (FFT), wavelet analysis including discrete wavelet transform (DWT) and so on.

Processing circuitry 38 may select a first frequency band of the two or more frequency bands and measure the one or more characteristics of the received signals. For example, processing circuitry 38 may measure a time-domain peak-to-peak voltage of the received post-ablation bioelectrical signal in the first frequency band. Based on the measured peak-to-peak voltage of the second bioelectrical signal in the first frequency band, processing circuitry 38 may estimate an efficacy of the cardiac lesion, e.g., an estimate of the ability of the formed or forming cardiac lesion to block or attenuate unwanted cardiac tissue conduction. In some examples, the estimate may be in the form of a lesion durability index, which, for example, processing circuitry 38 may cause to be displayed on user interface 52.

In some examples, the estimate of the cardiac lesion may include an estimate of long-term, e.g., chronic efficacy of the lesion. The generator may display a lesion durability index based on the frequency analysis of the iEGMs and possible other physiological measurements (e.g. temperature, impedance, total iEGM signals) to the operator. In some examples, the lesion durability index may be less than a lesion durability index threshold. Then subsequent ablation energy may be delivered responsive to the estimated efficacy being less than an efficacy threshold, for example, the lesion durability index may be less than a lesion durability index threshold.

In some examples, the estimated efficacy may indicate that a change of electrode location or electrode configuration may be desirable. In some examples, changing the position of the electrode may include moving the electrodes of ablation catheter 10 to a second position relative to the target location and delivering, via the one or more electrodes, a subsequent application of ablation energy at the second position. Ablation system 11 may again collect bioelectrical, and other sensed signals, immediately after the subsequent application of ablation energy (PFA, RFA etc.) and again display an estimate of lesion efficacy, e.g., based on frequency domain analysis of the sensed signals, such as an iEGM. In other examples, changing the position may include removing the electrode and ending the ablation procedure, e.g., because the estimated efficacy indicates no further ablation treatment is needed. In some other examples, if the lesion durability index is not reaching a threshold, and PFA energy is delivered in a bipolar fashion, additional electrodes may be selected for PFA energy delivery on the catheter (e.g. additional ring electrodes). In other examples, additional pulse trains are delivered. In other examples, the operator may choose to increase the PFA energy delivery by increasing the voltage or by changing a different pulse wave.

In some examples, system 11 may detect, analyze the frequency band automatically and automatically adjust the subsequent ablation energy delivery. For example, processing circuitry 38 may add or change electrodes, increasing voltage, the number of pulses in a pulse train or other predetermined automatic adjustments. In some examples, in response to the received bioelectrical signals, processing circuitry 38 may stop energy delivery automatically. In other examples, system 11 may detect and analyze the bioelectrical signals and display indications to the operator, e.g., via user interface 52. In some examples, the displayed information may include information on a mapping and navigation system, e.g., of the heart anatomy, with the location and quality of the lesion.

In some examples, system 11 may include functionality to suggest locations to move the electrodes, or reconfigure the electrodes, to collect data on existing or recently created lesions. In other words, processing circuitry may indicate, e.g., via user interface 52, locations to move ablation catheter 10 to recollect signals to evaluate lesion efficacy and/or durability. In some examples, system 11 may suggest specific time frames, e.g., a specified duration post-ablation to relocate the electrodes to recollect iEGMs to evaluate lesions.

In some examples, ablation system 11 may collect and store bioelectrical signals, such as an iEGM, over a wide frequency range, divide the collected and stored signal into frequency bands and analyze the signals in two or more frequency bands to estimate the lesion efficacy. In other words, processing circuitry 38 of ablation system 11 may estimate the lesion efficacy, such as by calculating a lesion durability index, based on the measured bioelectrical signal in the first frequency band and in a second frequency band. In some examples, processing circuitry 38 may estimate lesion efficacy based on measurements, such as peak-to-peak voltage, in three or more frequency bands. In some examples, estimates of lesion efficacy, e.g., a lesion durability index, may also be based on signals from other sensors, such as a temperature, an impedance, a pressure, thoracic impedance, cardiac rhythm, a blood chemistry measurement, an echocardiogram and so on. In some examples, the estimated efficacy of the lesion may be based on signals in the two or more frequency bands collected at the same time or approximately the same time. In other examples, the estimated efficacy may be based on signals received at different times, such as at a second time that is a predetermined duration subsequent to receiving the first bioelectrical signals.

In some examples, the frequency domain analysis may include comparing two or more signals. For example, processing circuitry 38 may perform a frequency domain analysis on the received baseline bioelectrical signal, e.g., the pre-ablation signal, or signals. The frequency analysis may include dividing the received pre-ablation bioelectrical signal into the same two or more frequency bands as for the received post-ablation signals.

Processing circuitry 38 may compare one or more characteristics of the received signals. In some examples the one or more characteristics may include a time-domain peak-to-peak voltage of the received baseline bioelectrical signal in the first frequency band to the time-domain peak-to-peak voltage of the received second post-ablation bioelectrical signal in the same first frequency band. Processing circuitry 38 may estimate the efficacy of the cardiac lesion based on the comparison, e.g., the comparison may be part of a lesion durability index calculation. In some examples the comparison of the signals in this disclosure may include a ratio of the amplitudes, comparing a frequency change, defining morphology change and similar comparisons. In some examples, the comparison may include some combination of signals. For example, processing circuitry 38 may compare characteristics of some combination of baseline signals to a similar combination of post-ablation signals, as well as some combination of signals in one or more frequency bands to some combination of signals in other frequency bands.

In some examples, post-ablation signals in this disclosure may refer to measured signals during or immediately after application of ablation energy, e.g., as soon as sensing circuitry can detect bioelectrical signals. In other examples, post-ablation signals may be measured after waiting for a predetermined duration, e.g., a few seconds, a minute, five minutes, thirty minutes and so on. In this disclosure, post-ablation signals measured after a predetermined duration will be referred to as “recovery signals.” In some examples, processing circuitry 38 may perform a frequency domain analysis on the received recovery bioelectrical signals. For example, the frequency domain analysis may include dividing the received recovery bioelectrical signals into the same two or more frequency bands as described above for the pre-ablation and post-ablation signals. In some examples, processing circuitry 38 may estimate lesion efficacy based on a comparison of recovery bioelectrical signals to either or both of the pre-ablation and post-ablation signals in any one or more frequency bands. In other examples, the estimated lesion efficacy may be based on comparison of characteristics of the recovery signal to a threshold value, such as a threshold amplitude. In some examples, the processing circuitry may calculate the second threshold value based on the baseline bioelectrical signal.

For any of the analysis of two or more frequency bands, the frequency bands may overlap in some examples. In other examples, a second frequency band may be distinct, e.g., separate from a first frequency band, and the second frequency band may include higher frequencies than the first frequency band.

In some examples, ablation system 11 may include instructions executable by processing circuitry 38 to perform analysis of patient specific frequencies and apply artificial intelligence (AI), including machine learning (ML), methodologies. In some examples, ablation system 11 may learn the patient specific frequencies and other measured characteristics, and optimize a response or a recommendation for a response. Processing circuitry 38 may apply the optimized sensing to future frequency sensing events in the same procedure. In some examples the future sensing events are within a few minutes of the initial measurements, while in other examples, future sensing events for the patient may be hours, days or weeks later.

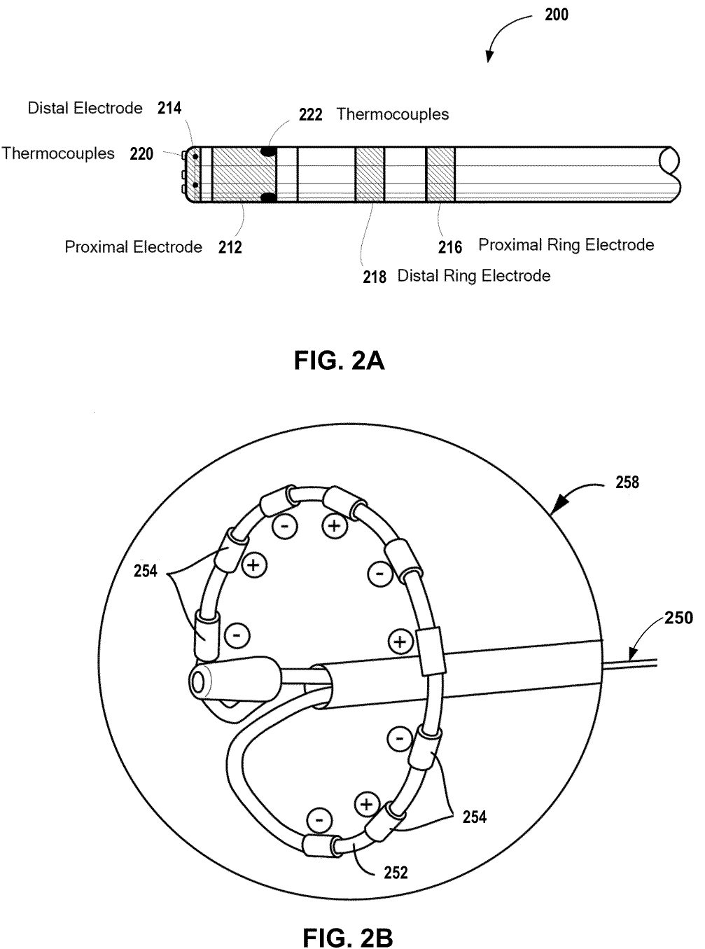

FIGS. 2A and 2B are conceptual diagrams illustrating example ablation catheters. Ablation catheters 200 and 250 are each examples of ablation catheter 10 described above in relation to FIG. 1. Components of ablation catheters 200 and 250 may have the same or similar characteristics and functions as described above for ablation catheter 10.

FIG. 2A illustrates one example ablation catheter 200 that can be used for PFA and/or RF ablation and measurement. In the example of FIG. 2A, catheter 200 includes a proximal electrode 212, a distal electrode 214, a proximal ring electrode 216, a distal ring electrode 218, distal thermocouples 220 and proximal thermocouples 222. In other examples, ablation catheter 200 may include one or more other sensors not shown in FIG. 2A.

Tissue electrical activity may be recorded as measured between the distal electrode 214 and one or more second electrodes 212, 216 or 218, for example, or any combination thereof, e.g., a unipolar configuration. Tissue electrical activity may also be recorded between any of the catheter electrodes (212, 214, 216, and 218) and a common reference such as Wilson’s Central Terminal. Catheter distal and proximal thermocouples 220 and 222 may sense temperature. In the example of FIG. 2A, the visible thermocouples 220 and 222 may be two of three thermocouples, with the third thermocouple not visible in FIG. 2A.

Power, voltage and/or current may be measured between the proximal and distal electrodes 212 and 214, for example. From measurements of voltage and current, processing circuitry of an ablation system, e.g., ablation system 11 described above in relation to FIG. 1, may calculate a magnitude and phase angle of impedance. The processing circuitry may receive signals from the distal and proximal thermocouples 220 and 222 indicating a temperature of the electrode location. Signals from the tip electrodes (proximal electrode 212 and distal electrode 214) and a voltage reference level from a return electrode, such as unipolar electrode 26 depicted in FIG. 1 may provide intracardiac electrograms 28 of FIG. 1, which processing circuitry may receive. The intracardiac electrograms (iEGMs) may include bipolar and/or unipolar intracardiac electrograms as well as monophasic action potentials. Signals from the tip electrodes 212 and 214, along with a voltage reference level from a return electrode (not shown in FIG. 2A) impedance measurements.

In some examples, the tip of catheter 200 may include features to shunt away from tissue being ablated. In some examples, catheter 200 may be configured to provide irrigation, e.g., saline injected through the catheter during ablation. As described above in relation to FIG. 1, bioelectrical signals via one or more electrodes 212, 214, 216, and/or 218, when received by processing circuitry of the ablation system may provide an indication of impedance, electrical properties (e.g., iEGM and/or MAP recordings) of the cardiac tissue proximate to the target lesion site. From the received signals, the processing circuitry may predict short term and chronic lesion efficacy, as described above in relation to FIG. 1.

FIG. 2B is a conceptual diagram illustrating a second example ablation catheter. As with ablation catheter 10 and 200 of FIGS. 1 and 2A respectively, catheter 250 may be configured to pass through a patient’s vasculature and be positionable proximate to a target tissue region for diagnosis or treatment. Catheter 250 may include a proximal portion (not shown in FIG. 2B) and a distal portion 258. In some examples, as described above in relation to FIG. 2A, catheter 250 also may include one or more lumens disposed within catheter 250, which may provide mechanical, electrical, and/or fluid communication, e.g., saline irrigation, between the proximal portion of catheter 250 and the distal portion 258. The distal portion 258 may generally define the one or more treatment region(s) configured to monitor, diagnose, and/or treat a portion of a patient, as described above in relation to FIG. 1. The treatment region(s) may have a variety of configurations to facilitate such operation. In the case of bipolar pulsed field delivery, distal portion 258 may include electrodes 254 that may be configured to form the bipolar configuration for energy delivery where energy passes between one or more electrodes and one or more different electrodes on the same electrode array, as described above in relation to FIG. 1. In an alternate configuration, one or more of electrodes 254 may serve as one pole while a second device containing one or more electrodes (not pictured) would be placed to serve as the opposing pole of the bipolar configuration.

In some examples, distal portion 258 may include an electrode carrier arm 252 that can transition between a linear configuration and an expanded configuration in which the carrier arm 252 has an arcuate or substantially circular configuration. The carrier arm 252 may include the plurality of electrodes 254 (for example, nine electrodes 254, as shown in FIG. 2B) that are configured to deliver pulsed-field energy. Carrier arm 252 when in the expanded configuration may lie in a plane that is substantially orthogonal to the longitudinal axis of the catheter 250. The planar orientation of the expanded carrier arm 252 may facilitate placement of electrodes 254 in contact with the target tissue. In some examples, electrode carrier arm 252 may transition between a linear configuration (not shown in FIG. 2B) and the expanded configuration depicted in FIG. 2B.

In some examples, the techniques of this disclosure may include using two or more separate catheters, e.g., a catheter to deliver ablation energy and a separate diagnostic catheter. For example, place the first catheter to collect a baseline measurement. Then remove the diagnostic catheter, introduce an ablation catheter, and perform the ablation. Finally introduce either the same or a third and different diagnostic catheter to record the signals to be analyzed post-ablation.

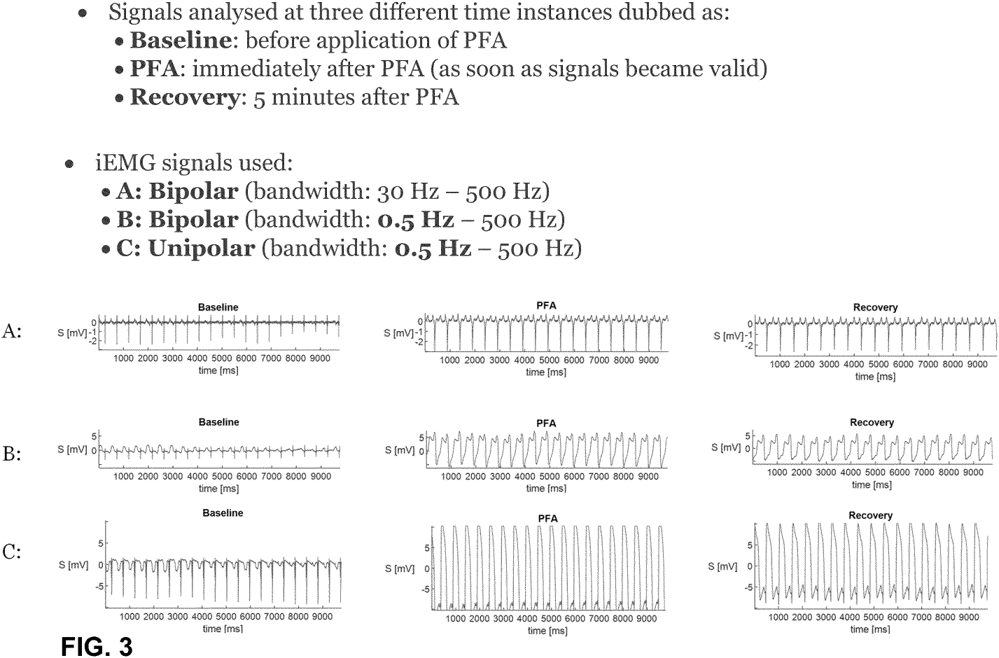

FIG. 3 includes time graphs illustrating example iEGM measurements before, immediately after and, in some examples, five minutes after ablation for both unipolar and bipolar configurations. As described above in relation to FIG. 1, electrodes on an ablation catheter may conduct bioelectrical signals received by processing circuitry of an ablation system. Processing circuitry may receive and store bioelectrical signals pre-ablation, or baseline, post-ablation, which in the example of FIG. 3 is indicated as “PFA,” and after a predefined duration, which in the example of FIG. 3 are recovery signals recorded about five minutes post-ablation.

The peak-to-peak amplitude for bipolar measurements appear to increase and change waveform shape (morphology) when comparing baseline to post-ablation and recovery signals for the 30 Hz to 500 Hz bandwidth (indicated by A). Including lower frequencies in the measurements (0.5 Hz to 500 Hz bandwidth) show a more significant increase in amplitude when comparing baseline to post-ablation and recovery measurements.

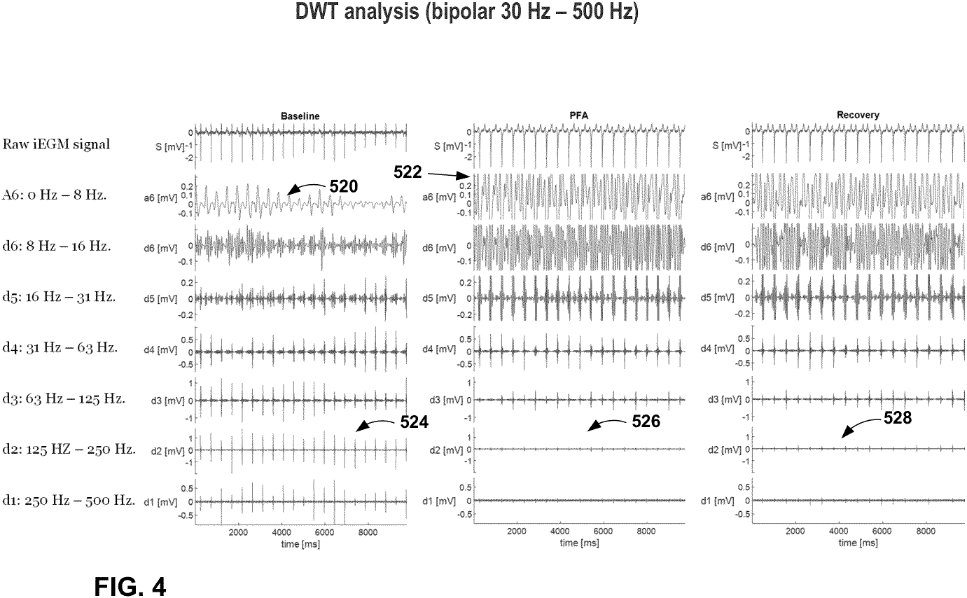

FIG. 4 includes time charts illustrating examples of discrete wavelet transformation decomposition of measured iEGMs before, immediately after and five minutes after ablation for a bipolar configuration with a clinical bandwidth of 30 - 500 Hz. As described above in relation to FIG. 1, processing circuitry of an ablation system of this disclosure may receive bioelectrical signals from electrodes on an ablation catheter and perform a frequency domain analysis on the received bioelectrical signal. In the example of FIG. 4, the frequency analysis includes dividing the received bioelectrical signal into multiple frequency bands, such as with a discrete wavelet transform, as shown in FIG. 4.

For the bipolar iEGM measurements of FIG. 4, excluding the lower frequencies, the low frequency band A6 (zero Hz to 8 Hz) shows a more than five-fold increase between baseline peak-to-peak amplitude 520 compared to post-ablation 522. In contrast, the higher frequency bands, d1 (250 Hz - 500 Hz) and d2 (125 Hz - 250 Hz) show a more than eight-fold decrease in peak-to-peak iEGM amplitude for baseline 524 to post-ablation 526 and recovery 528.

In other examples, an ablation system according to this disclosure may include sensing circuitry configured to filter the received bioelectrical signals into one or more frequency bands rather than decomposing the signal. In some examples, filtering the signal to analyze one or more specific frequency bands may reduce the signal processing steps performed by processing circuitry of the system. Recording and filter setting configured to detect and/or record only specific frequency band may provide rapid feedback into pulse generator without additional processing. Reducing the feedback time, when compared to first decomposing and analyzing the full spectrum of the received signal, may provide fast input for the system to take actions to prevent over damage to cardiac tissue, or other similar action. In other examples, the ablation system may include both the fast feedback filtering as well as full spectrum signal processing for more detailed post-ablation analysis.

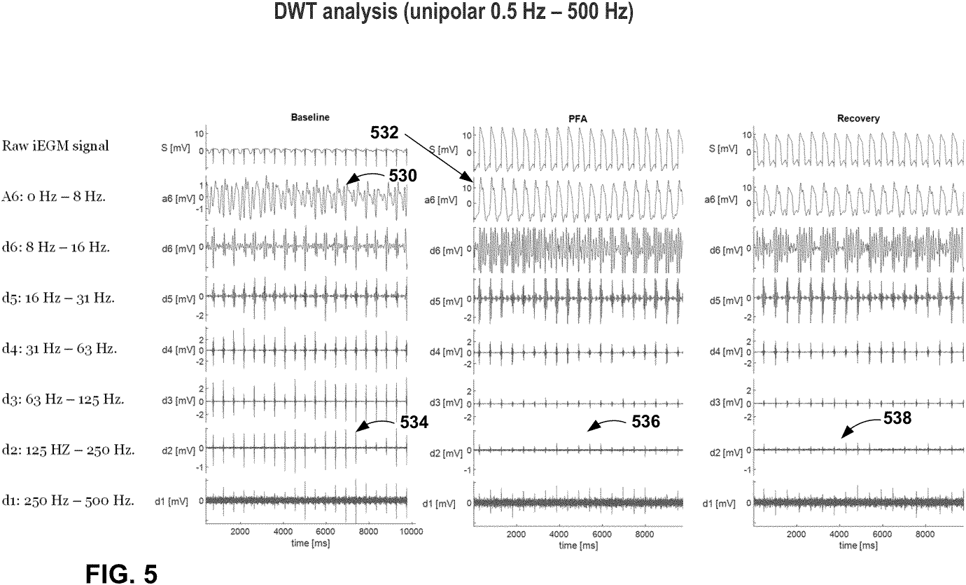

FIG. 5 includes time charts illustrating examples of discrete wavelet transformation analysis of measured iEGMs before and after ablation for a unipolar configuration (bandwidth 0.5-500 Hz). The example of FIG. 5, which includes the lower frequencies, e.g., less than 30 Hz, shows an even more significant increase, e.g., more than ten-fold increase in the A6 (0 Hz - 8 Hz) frequency band from baseline measurements 530 to post-ablation measurements 532. In the high frequency bands, e.g., d2 (125 Hz - 250 Hz) and d3 (63 Hz - 125 Hz), the peak-to-peak values show a decrease from pre-ablation 534 to post-ablation (536 and 538), but less of a change for the unipolar measurements of FIG. 5, compared to the bipolar measurements of FIG. 4 at the higher frequencies.

As described above in relation to FIG. 1, in some examples, processing circuitry of an ablation device of this disclosure may determine a threshold characteristic, e.g., a threshold amplitude based on the baseline measurement. The processing circuitry may provide an estimate of lesion efficacy based on comparing post-ablation bioelectrical signals to the threshold. For example, in the A6 frequency band, comparing signal 532 to a threshold of approximately 2-5 mV may provide an indication of the efficacy of the cardiac lesion. In other examples, the amplitude of the signal may increase by a different value, e.g., depending on the ablation equipment, patient, electrode location and other factors and the processing circuitry may determine a different threshold value. Similarly, comparing the decrease in amplitude found in signals 536 and 538, or other bioelectrical signals in the d3, d2 and d1 frequency bands may provide some indication of lesion efficacy. In other examples, the processing circuitry may compare the post-ablation signals to the baseline signals to estimate lesion efficacy. The processing circuity may use such comparisons to pre-ablation, post-ablation, and recovery signals or to a threshold, or any such combination of comparisons to calculate the lesion durability index.

The examples of FIGS. 4 and 5 depict frequency domain decomposition using discrete waveform transform (DWT), e.g., signal analysis, with Daubechies order six discrete wavelet. However, as described above in relation to FIG. 1, any type of frequency domain analysis may be used to isolate the different frequency bands. As noted above, in other examples, sensing circuitry may filter the received bioelectrical signals into one or more frequency bands rather than decomposing the signal as depicted by FIGS. 4 and 5. Additional detail on the DWT used for the examples of FIGS. 4 and 5 may be seen in the table below:

DWT Daubechies order 6
A6: 0 Hz-8 Hz.
d6: 8 Hz - 16 Hz.
d5: 16 Hz-31 Hz.
d4: 31 Hz - 63 Hz.
d3: 63 Hz - 125 Hz.
d2: 125 HZ - 250 Hz.
d1: 250 Hz - 500 Hz.

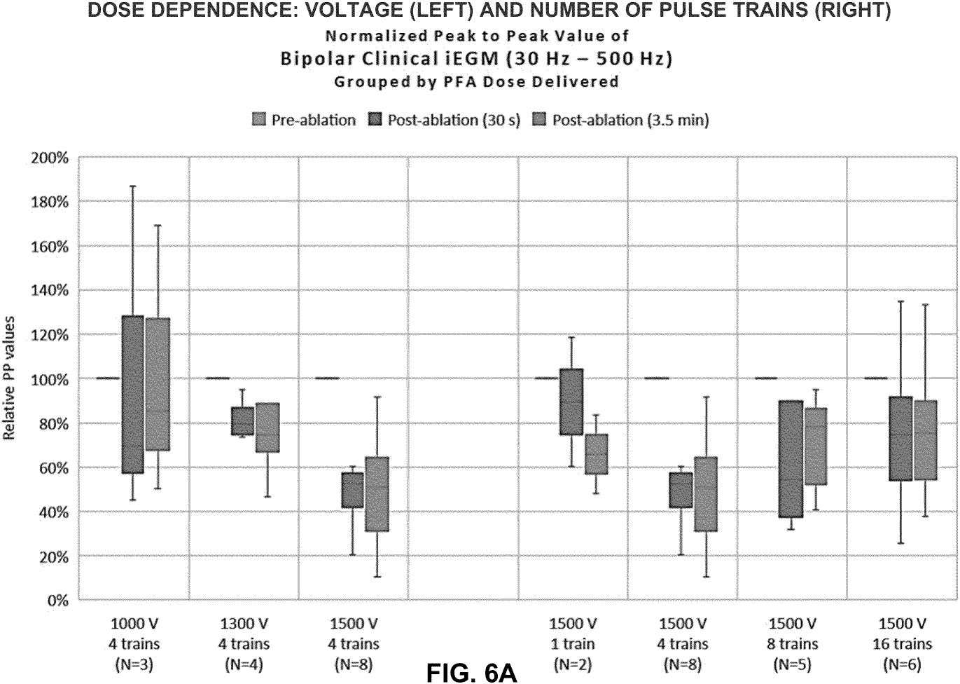

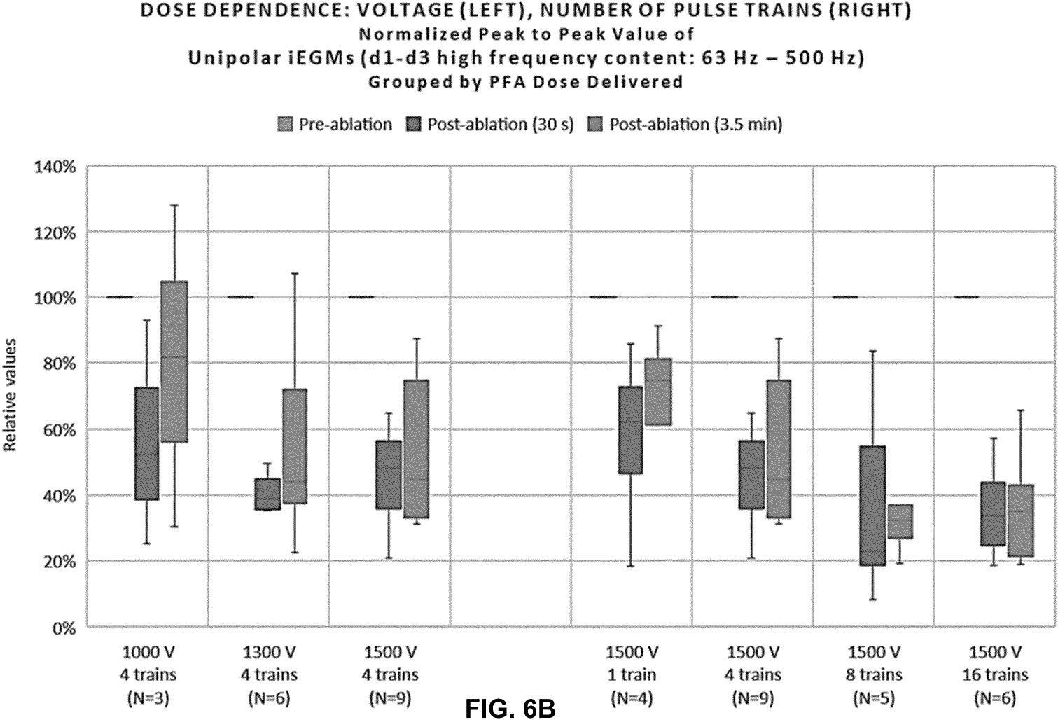

FIG. 6A is a chart illustrating the results of pulsed field ablation on measured bipolar relative peak-to-peak values over the clinical bandwidth (iEGM signals recorded at settings typical for clinical use). The chart depicts normalized peak-to-peak value measured in bipolar iEGM signals recorded over frequency range commonly used in clinical setting (e.g., 30 Hz - 500 Hz). The values are grouped based on the PFA dose delivered. In the example of FIG. 6A the PFA dose may be based on the number of pulse trains and/or the voltage delivered. The post ablation values were measured at both thirty seconds post ablation and again at three and a half minutes after ablation.

As the ablation energy dosage increases, the cardiac tissue should develop larger and more defined lesions. As shown in the example of FIG. 6A, a single pulse train of ablation energy should have less effect on cardiac tissue than an application of ablation energy with multiple trains of pulses, e.g., four, eight or sixteen trains. While it is believed that the dosage amount has an impact on the size, shape and permanency of a lesion, FIG. 6A shows that an increased dosage appears to have no clear dose dependence, e.g., the number of pulse trains, on the pre and post ablation measured peak-to-peak values for bipolar measurements, for the frequency band excluding the lower frequencies (e.g. clinically used iEGMs). This could be due to the overlapping effect; while there may be more than an eight-fold decrease in measured peak-to-peak values observed for the high frequency (d1 - d3) bands, there may be more than a five-fold increase in the low frequency (a6) band and these two opposite changes may cancel each other out to a large degree in the whole signal and thus mask the dose-dependency of the ablation effects.

FIG. 6B is a chart illustrating the results of ablation on measured relative peak-to-peak values in unipolar iEGM signals for the high frequency (e.g., 63 Hz -500 Hz) bands. The chart depicts normalized peak-to-peak values of the high frequency content (obtained with DWT decomposition) of the unipolar iEGMs. As with FIG. 6A, the values are grouped based on PFA dose delivered, e.g., number of pulse trains and/or voltage delivered. The unipolar measurements show a clearer dose dependency trend in higher frequency bands (e.g., d1 - d3), when compared to the clinical bipolar measurements (full frequency content: 30 Hz - 500 Hz) (FIG. 6A).

In more detail, FIG. 6A depicts bipolar signals, recorded with filters set to 30-500 Hz (as may be used in clinical setting, hence the label “clinical bipolar iEGMS”). Peak to peak value of individual cardiac signal was measured on the whole signal with no decomposition (thus mimicking the clinical practice where a clinician may estimate visually this peak-to-peak value from the signal on the screen during the procedure.).

In contrast, FIG. 6B depicts unipolar signals, rather than bipolar signals, recorded with filters set to 0.5-500 Hz (e.g., a broadened frequency range compared to the “clinical” range). Peak-to-peak value of individual cardiac signal was measured on the DWT-decomposed signal but only for the high frequency component, which is combination of components d1-d3 from the decomposition, and corresponds to frequency range of 63-500 Hz. That is FIG. 6B depicts only the relatively higher frequency component not the whole signal.

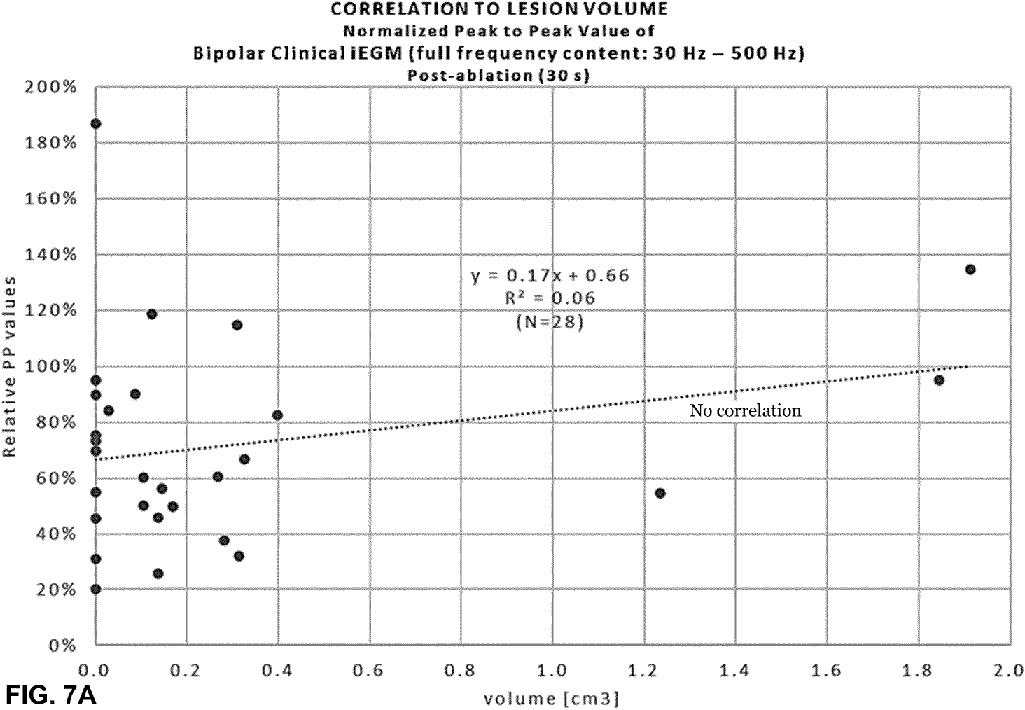

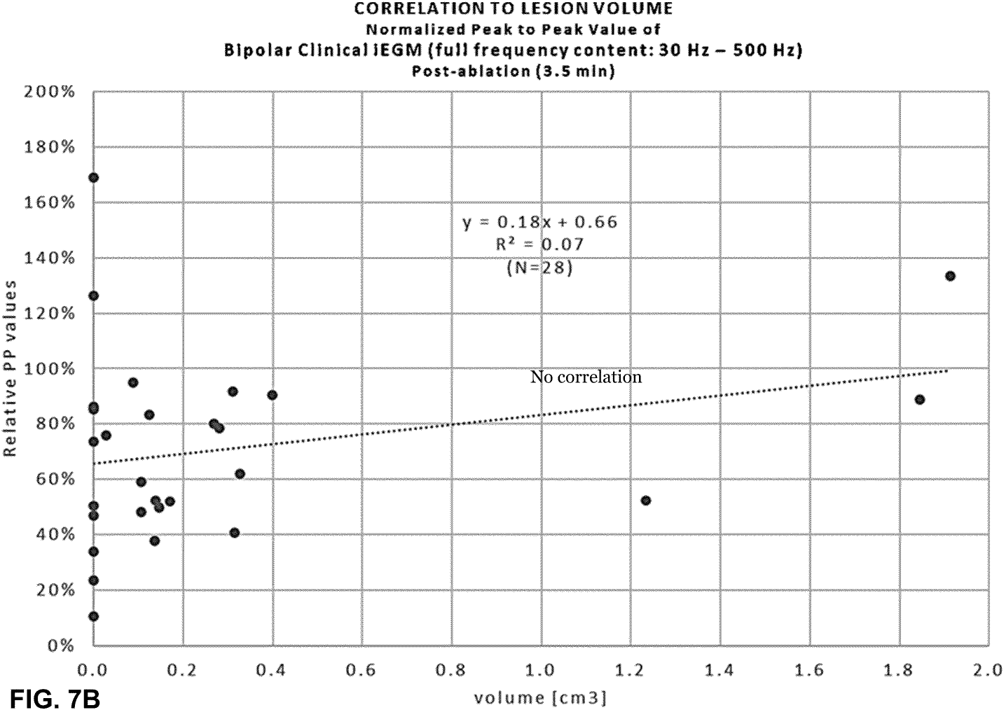

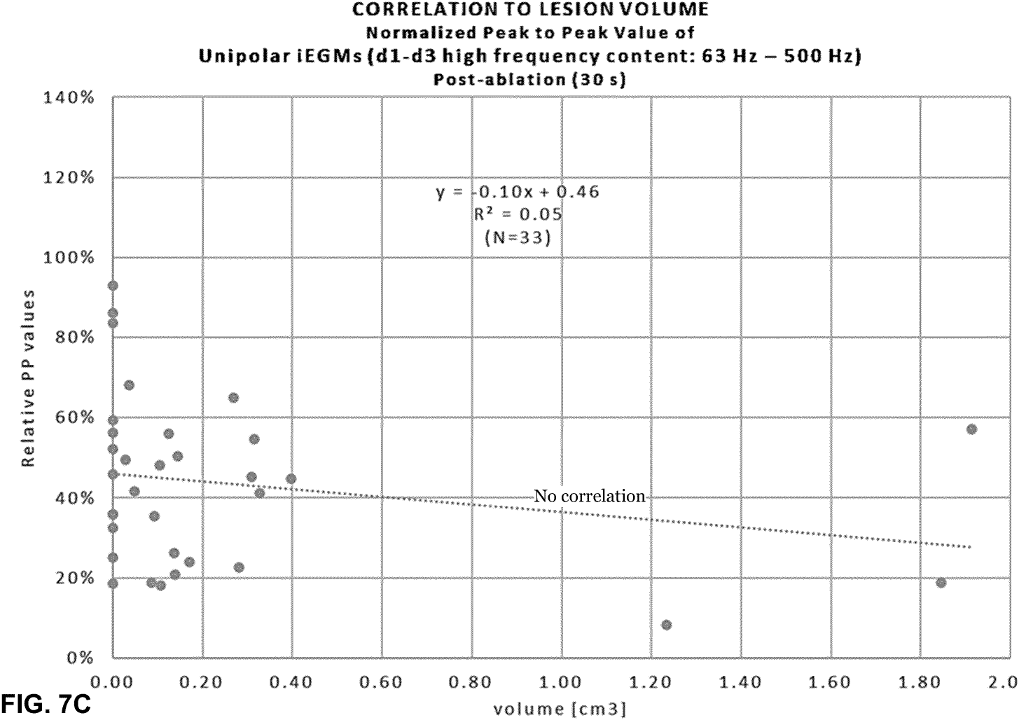

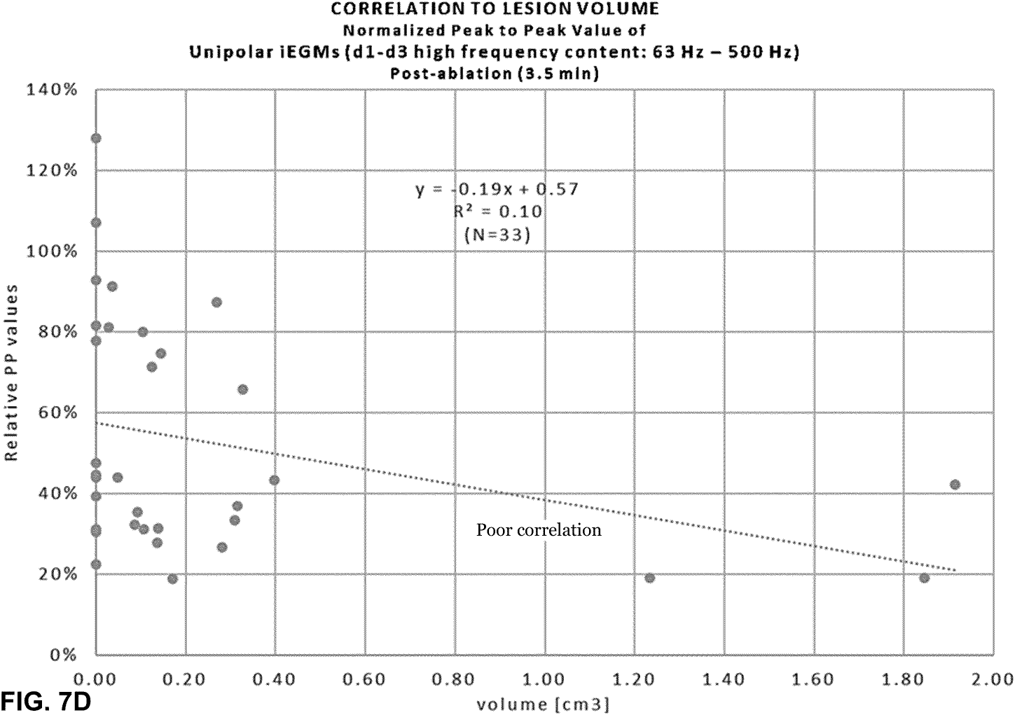

FIGS. 7A and 7B are charts illustrating an amount of correlation between measured lesion volume (assessed from gross pathology 6-7 weeks post-ablation) and measured bipolar peak-to-peak values at 30 seconds post ablation and 3.5 minutes post ablation over the wide clinically used bandwidth. As described above in relation to FIGS. 4 and 5, though the lesion volume may increase with increasing ablation dose, the bipolar measurements of relative peak-to-peak values for the clinical bandwidth, excluding the lower frequencies of less than 30 Hz (in clinical setting, the high pass filter for bipolar iEGM recording is typically set to 30 Hz), does not appear to correlate to lesion volume, and therefore such bipolar measurements may be less valuable in predicting chronic lesion efficacy.

FIG. 7A illustrates that data derived from clinical iEGM signals (bipolar signals with the “clinical” frequency range) does not correlate with the final clinical outcome (lesion size). In addition, the correlation, however poor, appears to be positive, which is the opposite of what would be expected. FIG. 7B also illustrates that data derived from clinical iEGM signals (bipolar signals with regular “clinical” frequency range) does not correlate with the final clinical outcome (lesion size). In addition, when comparing FIGS. 7A and 7B it can be seen that extending the time from 30s post ablation to 3.5 minute post ablation does not improve the ability to predict the lesion size from peak-peak amplitude based on clinical iEGM.

FIGS. 7C and 7D are charts illustrating an amount of correlation between measured lesion volume (assessed from gross pathology 6-7 weeks post-ablation) and measured unipolar peak-to-peak values at 30 seconds post ablation and 3.5 minutes post ablation for the high frequency (63 Hz - 500 Hz) bands. The high frequency content for the measured unipolar iEGM signals recorded post ablation over a frequency range (as opposed to the full frequency content of clinical bipolar iEGM signals from FIGS. 7A and 7B) still correlates poorly with the lesion size. Even though as seen in FIGS. 6A and 6B, that the higher frequency bands of unipolar signals show a more significant decrease in peak-to-peak amplitude from pre-ablation to post-ablation than the full frequency content of clinical bipolar signals, the higher frequency unipolar peak-to-peak measurements do not appear to be a valuable predictor of lesion volume as may be seen by (FIGS. 7A, 7B, 7C and 7D).

Although these figures depict data taken at 30 seconds and 3.5 seconds post-ablation, in other examples, any time interval may be used to collect data. For example, other intervals after treatment to be used for analysis of the signals, e.g., with respect to the pretreatment (pre-ablation) signals or with respect to some “universal” predetermined threshold parameters. The post ablation signals may be recorded either during the ablation procedure (continuous recording of the signals before, during, after ablation) or during a repeated procedure (remap) at later time (days, weeks, months... after the ablation procedure). In the first case (continuous recording) the time interval after the ablation may be measured in minutes, while for longer term measurements the time interval be in days, weeks, or similar intervals.

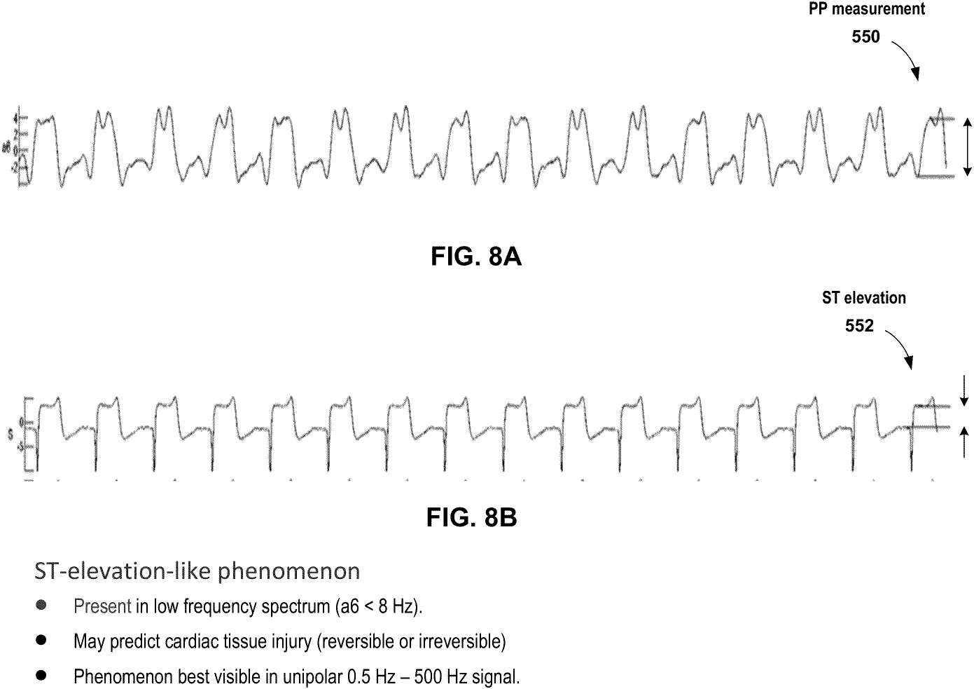

FIGS. 8A and 8B are time charts illustrating a post-ablation ST-elevation-like phenomenon as observed in the lower frequency band (0 Hz - 8 Hz) of the DWT-decomposed unipolar iEGMs recorded with the bandwidth setting of 0.5 Hz - 500 Hz. FIG. 8A shows one example of a DWT derived signal in the lower frequency band a6 (0.5 Hz - 8 Hz), while FIG. 8B shows and example of an original unipolar iEGM with bandpass filter set to a bandwidth of 0.5 Hz - 500 Hz before the DWT decomposition.

S-T elevation may be an indicator of early repolarization of the heart. In some examples S-T elevation is an indicator of ischemia and potential myocardial infarction. In other examples, S-T elevation is a normal variation in some people, which may be found, for example, in some male athletes. Cardiac ablation may result in early repolarization in some patients and therefore the iEGM of such patients may show S-T elevation. Some examples of transient repolarization abnormalities may mimic ischemia and may occur after elimination of overt pre-excitation. These repolarization abnormalities may not be due to cardiac injury and instead may be explained by the presence of cardiac memory. Note that S-T elevation, as seen in an externally measured electro-cardiogram (ECG), may differ from the S-T elevation like phenomena seen on an internally measured iEGM.

During “cardiac memory” the T-wave vector in sinus rhythm may align with the vector of the previous “abnormal,” and wide, QRS complex. Cardiac memory may present as a T-wave inversions (TWI), and in some examples, may be confused as ischemic T-wave changes, as noted above. Post-ablation repolarization abnormalities may resolve and disappear after a few hours. In some examples, follow-up ECGs demonstrated complete resolution of the T-wave changes in a minority of patients in the first one or two days after ablation. By three months, complete or near complete resolution of the temporary T-wave changes may occur in nearly all patients. However, S-T elevation may be a useful indicator for lesion formation.

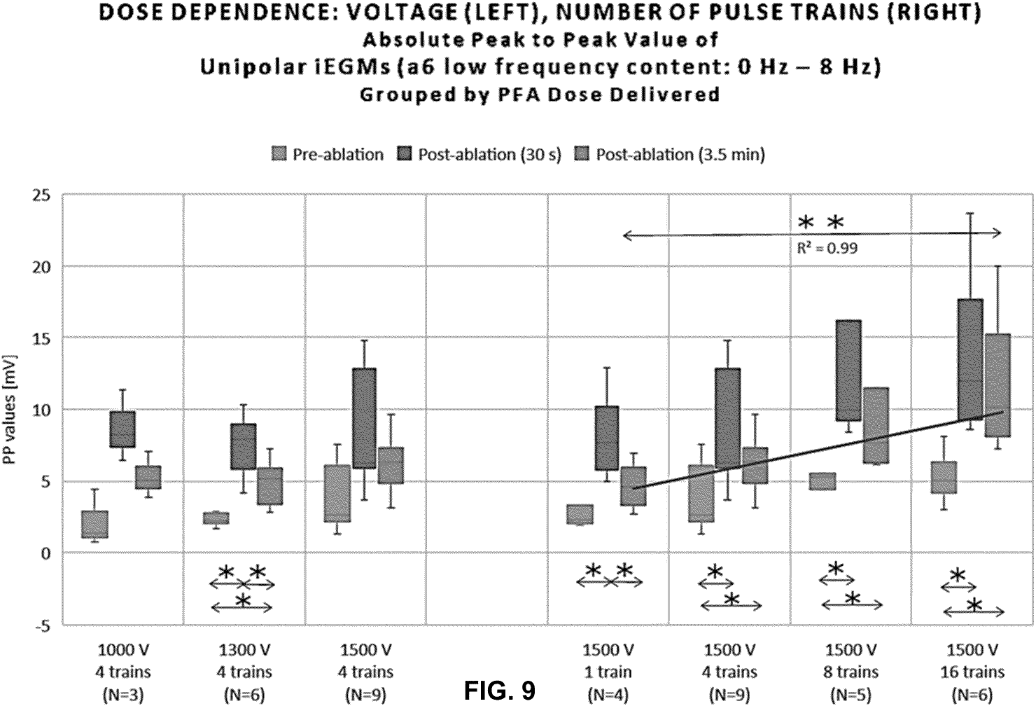

FIG. 9 is a chart illustrating the results of ablation on unipolar measured peak-to-peak values for the low frequency (0 Hz - 8 Hz) band. FIG. 9 depict dose dependence in the A6 band, e.g., low-frequency content. In other words, the graphs reflect predominantly the ST-elevation-like phenomenon observed in the unipolar signals. The post-ablation (30 s and 3.5 min) bars show absolute peak-to-peak values (in millivolts) instead of values normalized to baseline (pre-ablation values), because low-frequency content in the baseline signals was nearly negligible and could not be used reliably for normalization purposes as was the case for the high frequency content of the signals (presented in FIGS. 6A and 6B). FIG. 9 includes the same measured data as for FIG. 6B, except that FIG. 9 depicts only the low frequency component (component a6 from the DWT decomposition, corresponding to frequency range of 0-8 Hz).

The graph depicts the effect of changing dose, e.g., either four pulse trains at varying voltages, as well as the same voltage with varying number of pulse trains. The measured peak-peak values of low frequency component of the unipolar iEGM signals are significantly increased 30 seconds post ablation, but the dose dependence is better observed 3.5 minute post ablation, not within 30 seconds after ablation. This disclosure describes the “dose” as the number of pulse trains, or a change in voltage. However, dose in ablation may depend on other factors, such as the type of equipment, and the definition of dose should not be limited to only number of pulse trains or voltage. The final effect of the same “dose” (or ablation delivery protocol) can vary considerably from one treatment site to another, because it depends on other factors such as local tissue configuration and properties, positioning of the catheter and similar factors.

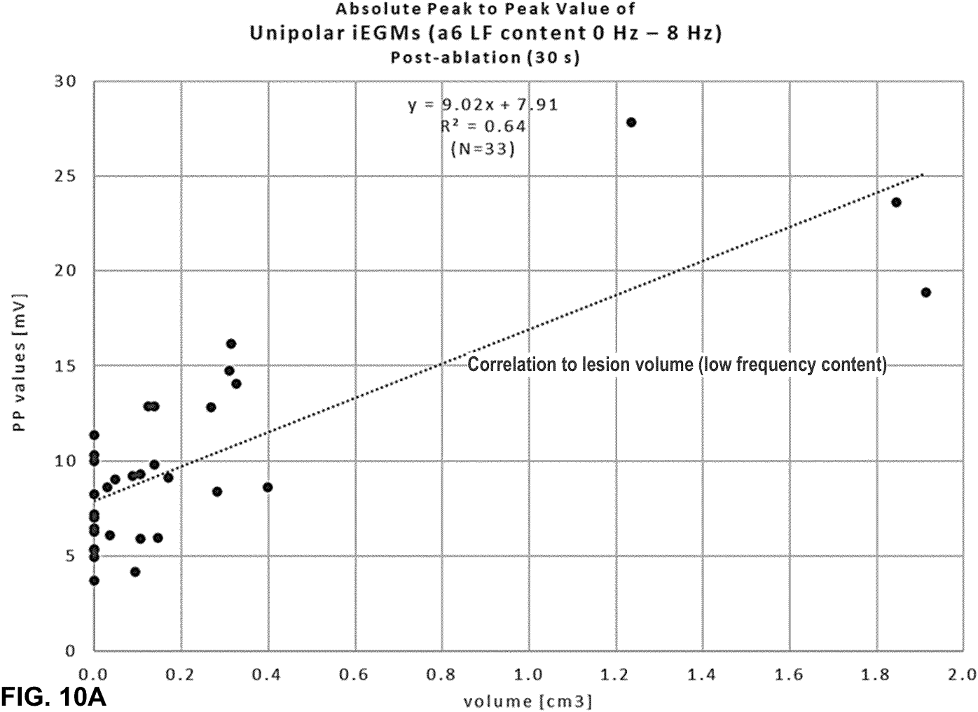

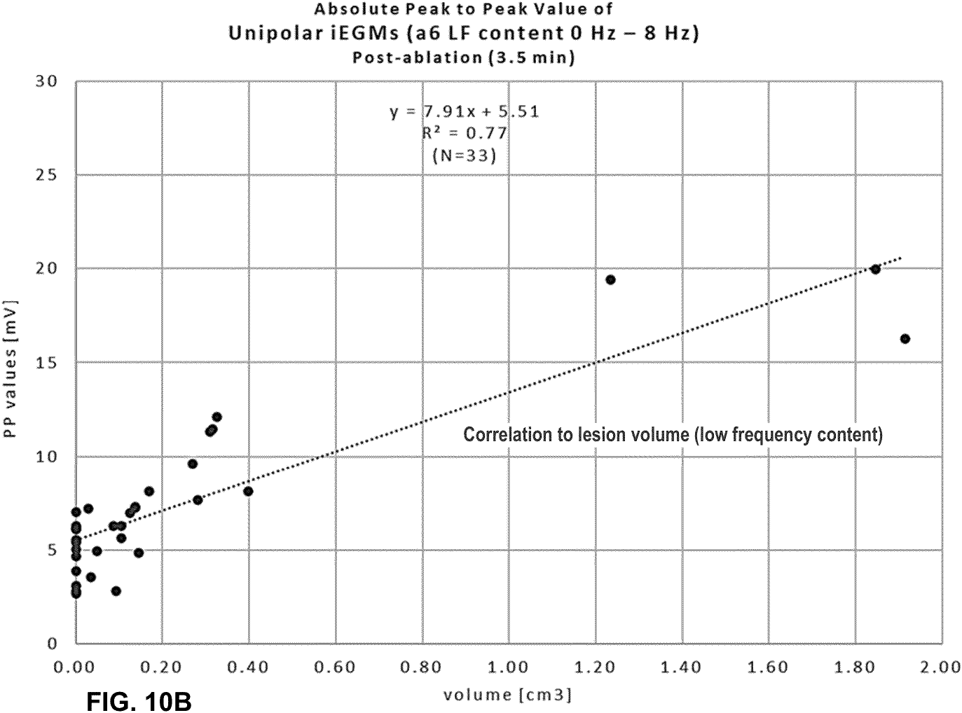

FIGS. 10A and 10B are charts illustrating an amount of correlation between measured lesion volume (assessed from gross pathology 6-7 weeks post-ablation) and measured unipolar peak-to-peak values for the low frequency (0 Hz - 8 Hz) band at 30 seconds and 3.5 minutes post ablation. The example of FIGS. 10A and 10B depict a correlation of measured signal parameters, e.g., characteristics of measured bioelectrical signal, to the measured lesion volume, which in the example of FIGS. 10A and 10B, is regardless of the dose, such as the number of trains of pulses or voltage delivered during PFA. The example of FIGS. 10A and 10B appear to show that the extent of the ST-elevation-like phenomenon (reflected in the low-frequency component of both bipolar (data not shown in FIGS. 10A and 10B) and unipolar signals recorded with 0.5 Hz highpass boundary frequency) correlates more strongly with lesion volume than the extent of decrease in the high-frequency components of these signals, which was also shown in FIGS. 4, 5, 6B, 7C, 7D and 9. This relatively strong correlation between the low frequency content of the unipolar iEGM signals recorded either 30 seconds or 3.5 minutes post ablation and the lesion size in FIGS. 10A and 10B is a stark difference to FIGS. 7B and 7D. In addition, the correlation is also significantly improved for FIG. 10B in comparison to FIG. 10A. The data shows that the correlation improves with time post ablation, e.g., the effect may be better observed 3.5 minutes post ablation than 30 seconds or less after the ablation.

Therefore, considering FIGS. 4 - 10B, changes induced by PFA (30 seconds after PFA and 3.5 minutes later), such as the decrease in the high-frequency content (disappearance of the near field signal) and the increase of the low-frequency content (the ST-elevation-like phenomenon) are statistically significant for all “doses” (different numbers of trains of pulses or voltages) except, in some examples, for the lowest “dose” of one pulse train. Peak-to-peak voltage measurements (bipolar iEGM signals, 30 Hz -500 Hz, typical clinical setting) do not indicate a clear dose-dependent trend (FIG. 6A) even though the effect of PFA is evident at all doses, but the differences between the effects of various doses are not statistically significant (p>0.05, ANOVA). Furthermore, there is no correlation to chronic lesion volume (FIGS. 7A, 7B). In this manner, peak-to-peak voltage measurement (bipolar 30 Hz - 500 Hz, typical clinical setting) may not be a useful predictor of lesion volume after PFA. The high frequency bands (63 Hz - 500 Hz) of unipolar iEGM signals indicate a dose dependency trend for the number of pulse trains (FIG. 6B) even though the differences between doses are not statistically significant (ANOVA, p>0.05), but no correlation to chronic lesion volume even after waiting for a predetermined duration and taking a recovery measurement (FIGS. 7C and 7D). However, peak-to-peak measurements in the low frequency range (0 Hz- 8 Hz) of unipolar iEGMs indicate a useful correlation to chronic lesion volume, even right after PFA (FIG. 10A) with the correlation improving further after waiting for a predetermined duration (FIG. 10B). For the low frequency range (0 Hz - 8 Hz) of unipolar signals the results indicate a dose dependent trend for the number of pulse trains (FIG. 9), but the difference is statistically significant only between the lowest and highest doses (p<0.05, ANOVA followed by post hoc pairwise comparisons). In this manner the ablation system, e.g., system 11 of FIG. 1, may estimate an efficacy of the cardiac lesion based on the measured peak-to-peak voltage of the post-ablation bioelectrical signal in the A6 frequency band. In some examples, the ablation system of this disclosure may further include analysis of other frequency bands, as well as other measurements from sensors, such as sensors 20 and sensors 46 described above in relation to FIG. 1, as part of a lesion durability index to predict the chronic efficacy of a cardiac lesion formed by an ablation procedure, such as PFA.

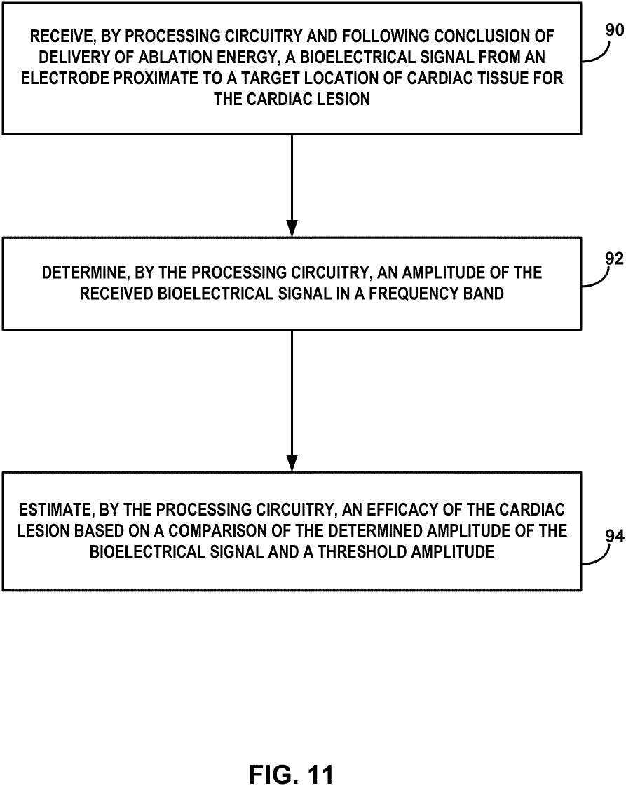

FIG. 11 is a flowchart illustrating an example operation of the lesion analysis system of this disclosure. The example of FIG. 11 describes one possible operation for evaluating a cardiac lesion formed by an ablation procedure. The blocks of FIG. 11 will be described in terms of FIGS. 1, 2A and 2B, unless otherwise noted. As seen in the example of FIG. 11, processing circuitry, such as processing circuitry 38 depicted in FIG. 1, initially may receive a bioelectrical signal from an electrode proximate to a target location of cardiac tissue for the cardiac lesion (90) following conclusion of delivery of ablation energy. In some examples, one or more electrodes on the ablation catheter, e.g., catheter 10, 200 or 250 may conduct the bioelectrical signal from cardiac tissue proximate to the one or more electrodes. In some examples the bioelectrical signal may be a bipolar signal in which two or more electrodes are proximate to the cardiac tissue. In other examples the bioelectrical signal may be a unipolar signal in which one or more electrodes are proximate to the cardiac tissue, and one or more other electrodes, e.g., unipolar electrodes 26, placed on the patient’s skin, or other locations on the patient to also conduct the bioelectrical signal to the processing circuitry.

As described above in relation to FIG. 1, in some examples, the processing circuitry may receive the bioelectrical signal during or shortly after delivery of the ablation energy, e.g., as soon as the circuitry and cardiac tissue has recovered enough from the delivered ablation energy to provide bioelectrical signals that the sensors or various electrodes may sense. In other examples, the processing circuitry may receive the bioelectrical signals after waiting for a predetermined duration, such as a few seconds, a minute, five minutes, or any other duration. In some examples, the patient may return hours or days later to measure the bioelectrical signals.

Next, the processing circuitry may determine an amplitude of the received bioelectrical signal in a selected frequency band (92). In some examples, the frequency band may include a specified sub-band of a larger frequency spectrum. For example, the processing circuitry may determine the amplitude, or other characteristics such as power, frequency, pulse repetition rate, pulse width and other similar characteristics in a frequency band less than 30 Hz. In some examples, the processing circuitry may determine the amplitude of bioelectrical signals in a frequency band including zero to eight Hz, as described above in relation to FIGS. 9, 10A and 10B. In other examples, the processing circuitry may measure signal characteristics in one or more additional frequency bands, such as a frequency band that includes higher frequencies, such as 63 -500 Hz as described above in relation to FIG. 6B. In some examples the one or more additional frequency bands may be separate and distinct frequency bands. In other examples the frequency bands may overlap.

Next, the processing circuitry may estimate an efficacy of the cardiac lesion based on a comparison of the determined amplitude of the bioelectrical signal and a threshold amplitude (94). For example, as depicted in FIG. 5, the peak-to-peak amplitude of the signal in the A6 (0 Hz - 8 Hz) band may increase to greater than two millivolts (mV) after application of ablation energy. In other examples, the amplitude of the signal may increase by a different value, e.g., depending on the ablation equipment, patient, electrode location and other factors. In some examples, the processing circuitry may receive a baseline bioelectrical signal prior to the delivery of the ablation energy, e.g., signals 530 and 534 of FIG. 5. In some examples, the processing circuitry may determine the threshold amplitude based on the baseline bioelectrical signal. In other examples, compare the baseline bioelectrical signal to the bioelectrical signal received after the delivery of ablation energy, e.g., signals 532, 536 and 538 of FIG. 5 to estimate the efficacy of the cardiac lesion.

In other examples other sensors, e.g., sensor 20, including temperature, pressure or force and other types of sensors may provide a second bioelectrical signal to the processing circuitry to help evaluate the efficacy of the cardiac lesion. In some examples, the processing circuitry may use any one or more such bioelectrical to calculate a lesion durability index, as described above, which may provide a prediction of the clinical efficacy of the cardiac lesion. For example, if temperature or force increases are observed in a certain electrode, this indicates tissue contact, and can be used in conjunction with the iEGM frequency analysis to predict lesion efficacy.

In other example, a third and fourth bioelectrical signal may provide additional information to the processing circuity to evaluate lesion efficacy. For example, in addition an algorithm involving the change of frequency spectrum of iEGM signals, temperature, local impedance, and contact force following ablation can be integrated into an algorithm that provides predication of lesion formation. For example, a slight increase in electrode temperature, an increase in local contact force (measured via a contact force sensor), a decrease in local impedance, in conjunction with characteristic changes of frequency spectra of iEGM can provide an integrated algorithm to more accurately predict lesion efficacy. Additional bioelectrical signals may be added to such examples to make such algorithms more robust and reliable.

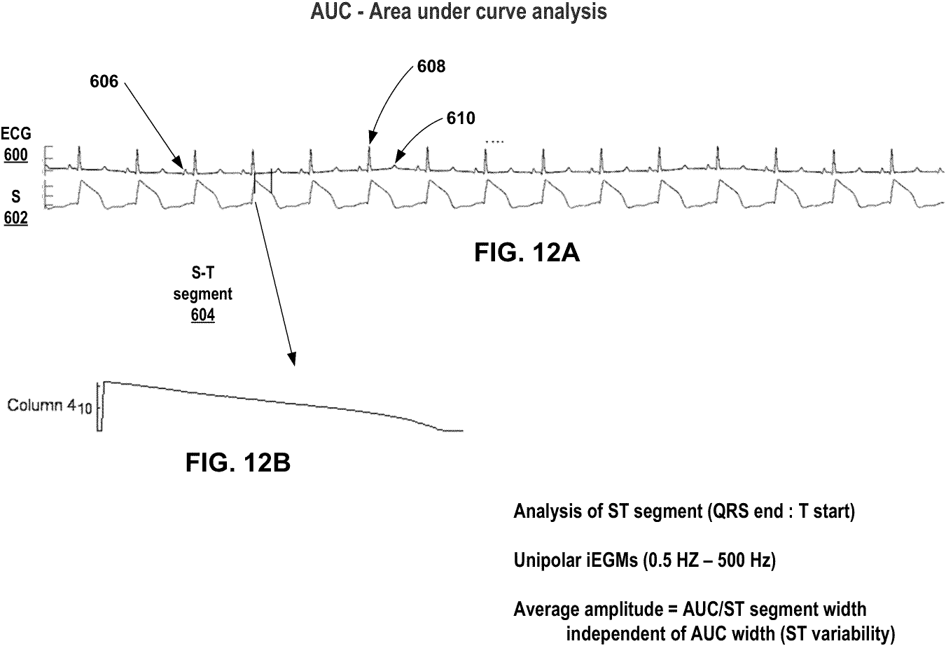

FIGS. 12A and 12B are time charts illustrating a post-ablation area under the curve (AUC) analysis as measured in time domain for the ST segment of the cardiac cycle. The time charts of FIGS. 12A and 12B describe one possible technique to measure the ST elevation described above in relation to FIG. 8B.

ECG 600 depicts the ECG for a series of consecutive heart beats, while S 602 depicts the unipolar iEGM signal (recorded in parallel with the ECG and other bioelectric signals) at the site of the ablation AFTER the ablation, showing the ST elevation-like phenomenon the post prominent feature of the whole signal . It is this part of the iEGM that is used to quantify the ST elevation-like phenomenon that is shown (for one heartbeat) in FIG. 12B. Two ways to quantify the ST elevation-like phenomenon are presented in this application: the one based on DWT (the A6 low frequency component with its peak-to-peak amplitude) and the one based on AUC and presented here.

ST elevation is something that cardiologists usually observe on the level of the whole heart (from ECG) and may be a result of regional ischemia (after infarct, for example). The example of FIGS. 12A and 12B however illustrates a morphologically similar effect, but on the level of highly localized part of the heart treated by ablation. That is the reason why this effect is referred to in this disclosure as ST elevation-like phenomenon (instead of ST elevation). Note in the example of FIG. 12A how the ECG signal (ECG 600) looks normal, e.g., there is no ST elevation on the level of the whole heart while there is a large ST elevation-like phenomenon present in the iEGM signal recorded locally at the site of ablation in the same very heart at the same time.

In the example of FIGS. 12A and 12B, the analysis of ST segment 604 included a measurement of unipolar iEGMs in the frequency range of 0.5 HZ - 500 Hz. The average amplitude in this example is defined as AUC of the ST segment divided by the ST segment 604 width, which makes this parameter independent of the ST width and its variability. The AUC analysis resulted in good correlation to the lesion size, depth, and stability.

Normalizing the ST segment AUC (area under curve of the ST segment) by dividing it by the ST duration makes this AUC parameter independent of the ST duration. This is useful, because the ST duration may vary, e.g., the ST duration depends on the heart rate, for example. AUC itself has no width (or duration), while the ST segment does. AUC is calculated by integrating the iEGM over the interval corresponding to the ST segment. Dividing the AUC by the ST segment duration, results in the average amplitude of the iEGM within the ST segment, a parameter independent of the duration of the ST segment. In this disclosure, this parameter is described as “average ST segment amplitude.”

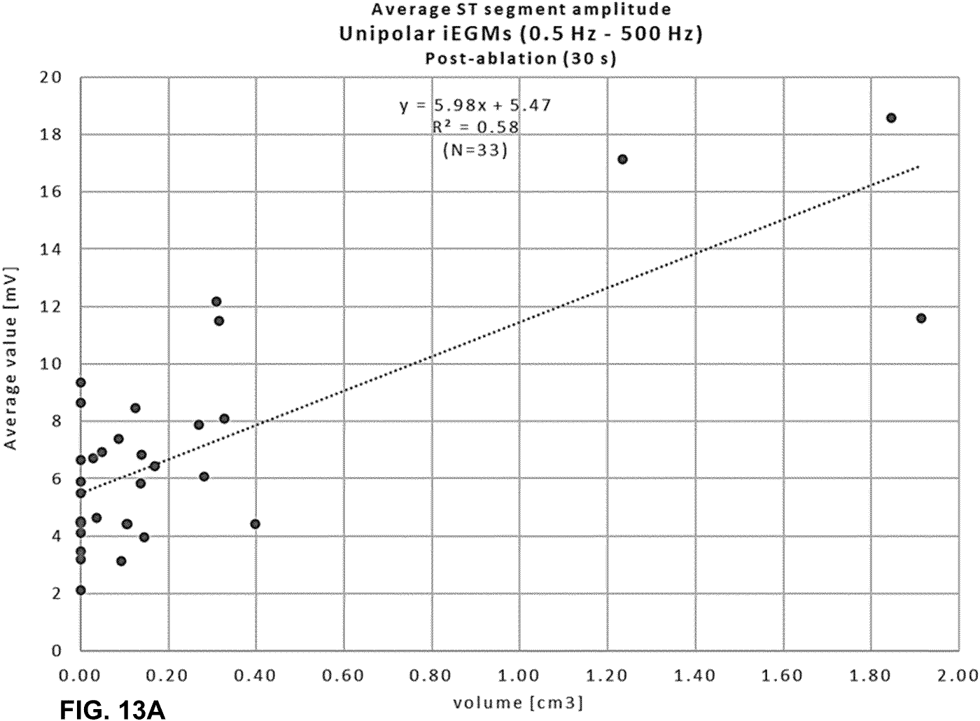

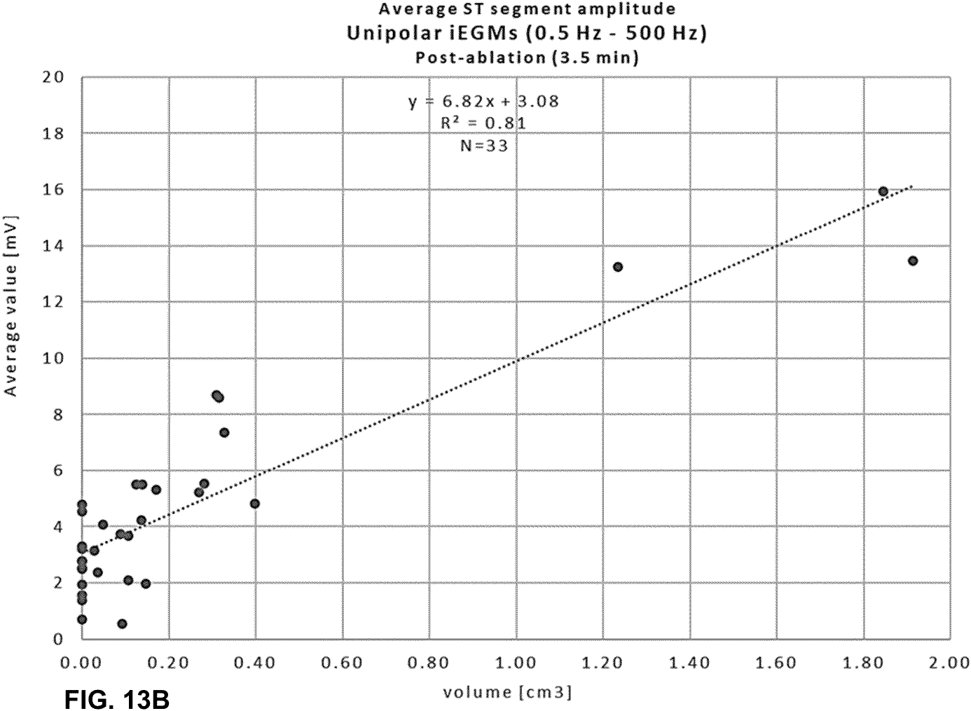

FIGS. 13A and 13B are charts illustrating the correlation of average ST segment amplitude to measured lesion volume based on unipolar iEGM measurements taken 30 seconds after ablation as well as 3.5 minutes after ablation. The charts of FIGS. 13A and 13B describe correlation between lesion volume and the measured average ST segment amplitude. FIG. 13A depicts measured average ST segment amplitude 30 seconds after ablation while FIG. 13B depicts the measurement 3.5 minutes after ablation. Waiting to take the post-ablation measurement may result in better correlation than measuring only a few seconds after ablation.

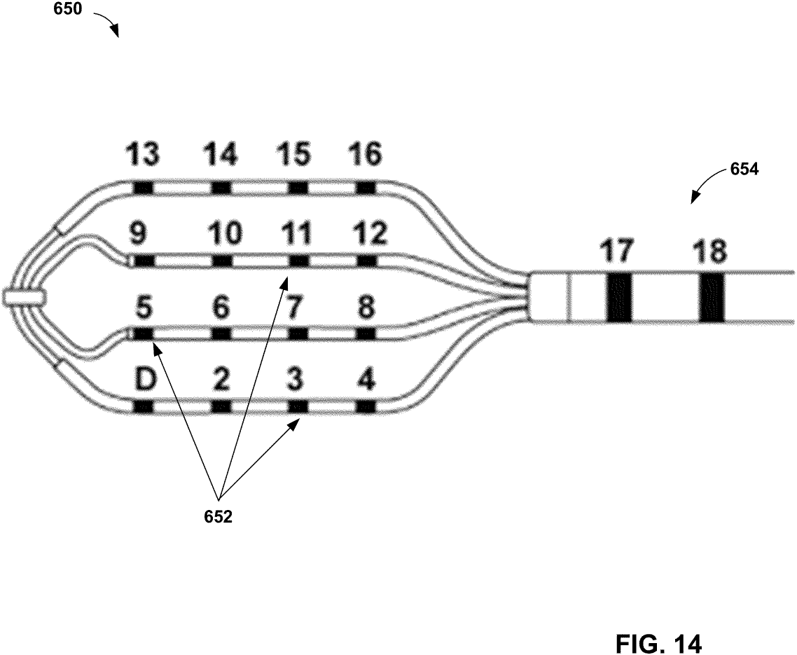

FIG. 14 is an example implementation of a grid catheter which may be used for measuring cardiac signals according one or more techniques of this disclosure. Grid catheter 650 may include electrodes 652 arranged in a grid pattern, as well as additional electrodes, e.g., 654 located along the lead. Grid catheter 650 is another example of the catheters described above in relation to FIGS. 2A and 2B.

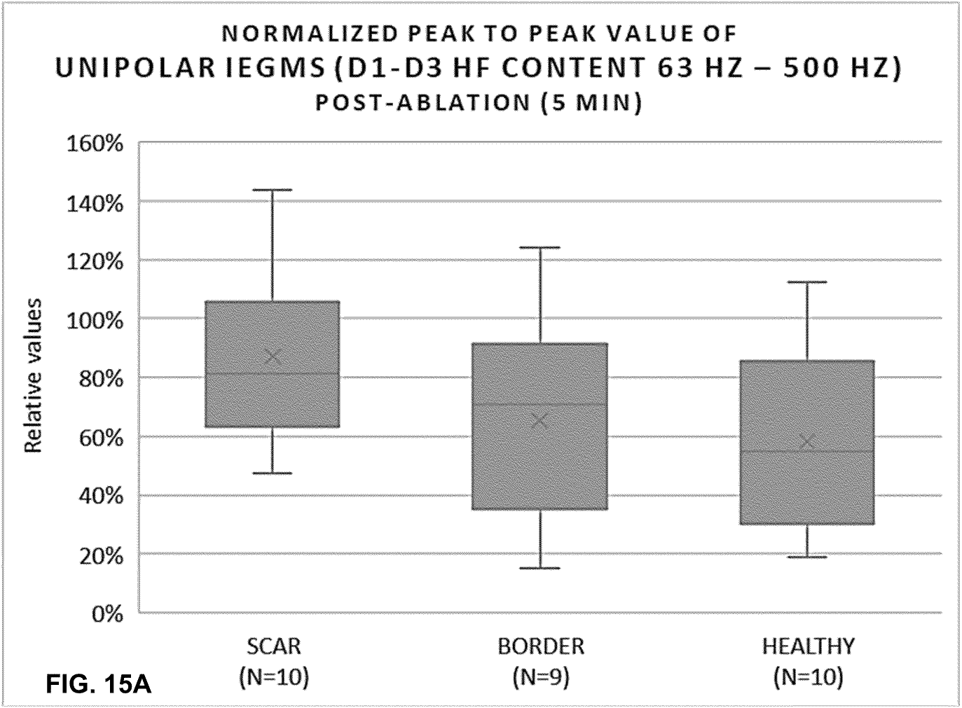

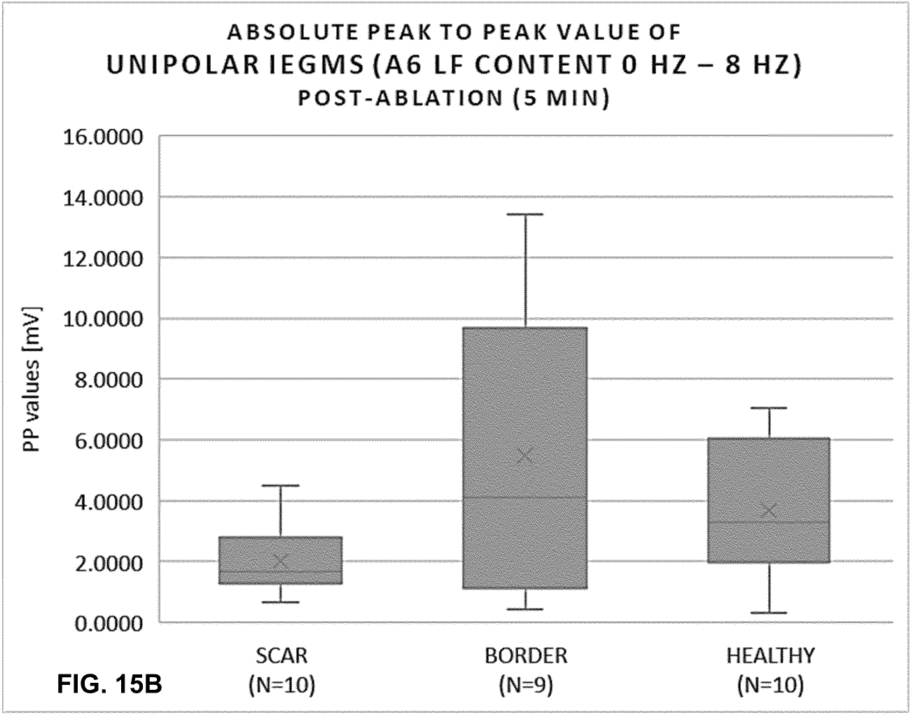

FIGS. 15A and 15B are graphs illustrating the impact of applying ablation in different regions with respect to its viability within the same heart, e.g., for the purpose of lesion homogenization. In the example of FIG. 15A, the measurements included peak to peak values at higher frequencies with a bandwidth of 63 - 500 Hz and FIG. 15B, the measurements included peak to peak values at lower frequencies with a bandwidth of 0 -8 Hz taken five minutes after ablation. The charts are based on unipolar iEGMs with a bandwidth of 0.5 - 500 Hz decomposed into frequency subbands using discrete wavelet transform. The locations of the measurement catheter include those on the scar tissue, on the border between the scar and healthy tissue and on healthy tissue. The measurements show less pronounced relative decrease in the peak-to-peak values in the higher frequency band and less ST elevation (low frequency band) in scar tissue.

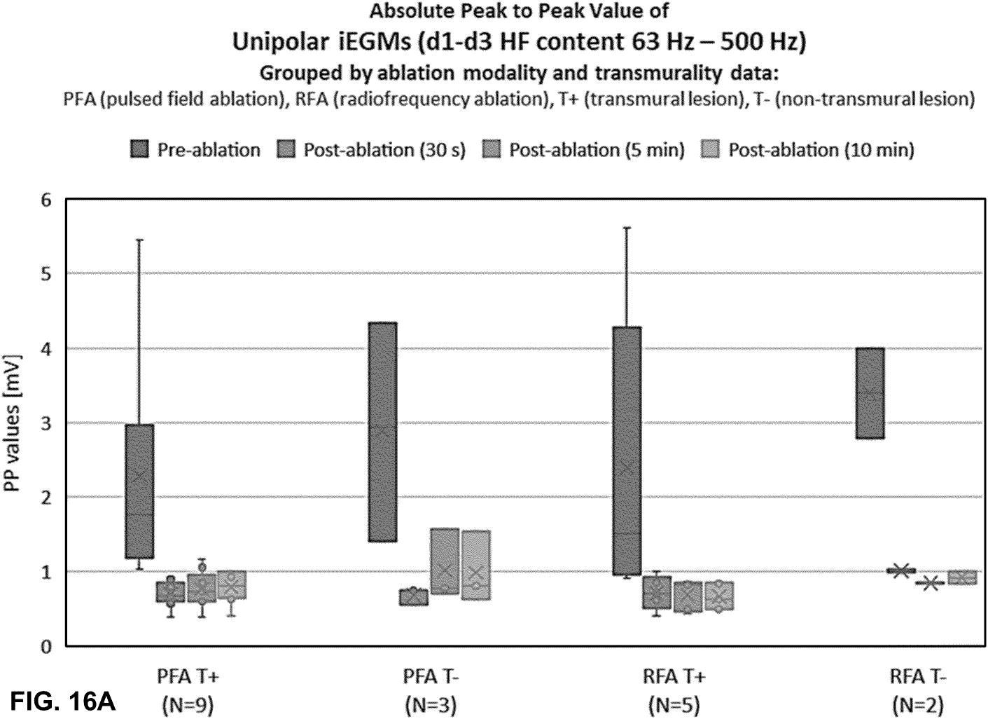

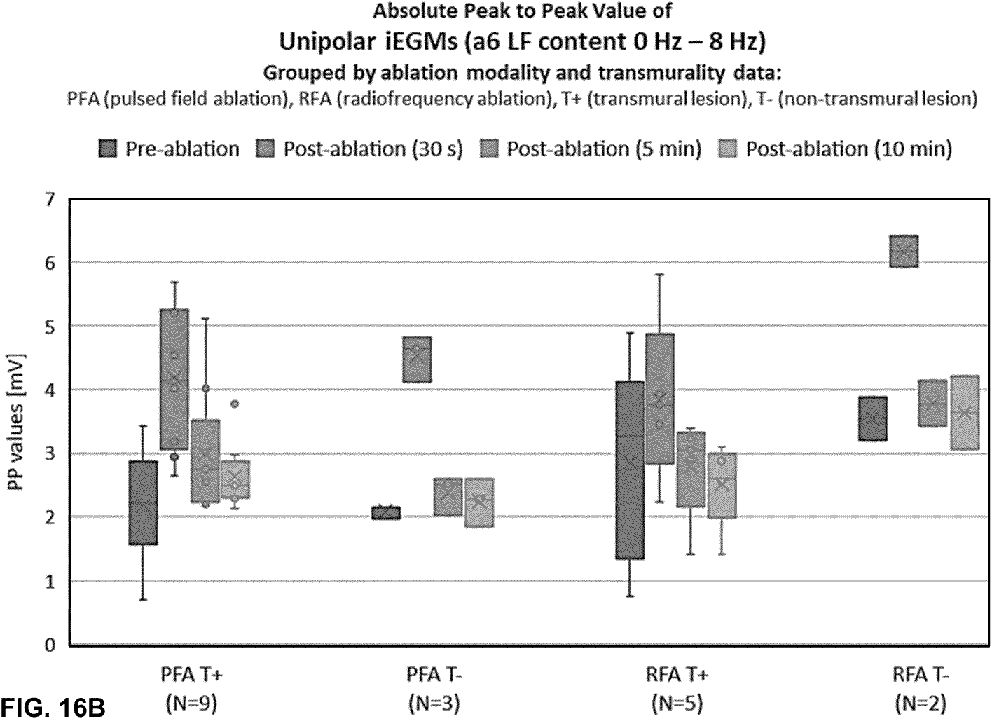

FIGS. 16A and 16B are graphs illustrating the impact of applying ablation in healthy atrial tissue, e.g., for pulmonary vein isolation. The results are presented separately for PFA and RFA ablation modalities and separately for transmural (T+) and non-transmural (T-) lesions (based on gross pathology examination of the hearts).

In the example of FIG. 16A, the measurements included peak to peak (PP) values at higher frequencies with a bandwidth of 63 - 500 Hz taken 30 seconds, five minutes, and ten minutes after ablation. For FIG. 17B, the measurements included peak to peak values at lower frequencies of 0 - 8 Hz taken 30 seconds, five minutes, and ten minutes after ablation. The charts in the examples of FIGS. 16A and 16B are based on unipolar iEGMs with a bandwidth of 0.05 - 500 Hz (as opposed to 0.5 Hz - 500 Hz for all previous charts). The data for the charts were decomposed with the same procedure using DWT as for all previous charts where decomposition was used.

The example of FIG. 16A shows that higher frequency content PP values decreased across all groups with no obvious difference in recovery dynamics. In the example of FIG. 16B, the lower frequency content PP values increased across all groups with observed difference in recovery dynamics for both transmural groups compared to both non transmural groups. For both transmural groups PP values return to baseline after five minute with no recovery dynamics from five minute to ten minute. However, for both transmural groups there is further recovery observed from five minute to ten minute. This distinction that may be useful to predict the transmurality of the lesion during the ablation procedure which may allow to increase the treatment to achieve transmurality in all lesions.

The techniques of this disclosure may also be described in the following examples.

Example 1: A method for evaluating a cardiac lesion formed by an ablation procedure comprising receiving, by processing circuitry and following conclusion of delivery of ablation energy, a bioelectrical signal from an electrode proximate to a target location of cardiac tissue for the cardiac lesion; determining, by the processing circuitry, an amplitude of the received bioelectrical signal in a frequency band of the received bioelectrical signal; and estimating, by the processing circuitry, an efficacy of the cardiac lesion based on a comparison of the determined amplitude of the bioelectrical signal and a threshold amplitude.

Example 2: The method of example 1, wherein the ablation energy is pulsed field ablation (PFA).

Example 3: The method of example 1 or example 2, wherein the frequency band comprises frequencies less than 30 Hz.

Example 4: The method of example 1-3, wherein the frequency band is 0 Hz to 8 Hz.

Example 5: The method of any of examples 1-4, wherein subsequent ablation energy is delivered responsive to the estimated efficacy being less than an efficacy threshold.

Example 6: The method of any of examples 1-5, wherein the received bioelectrical signal is a first bioelectrical signal, the method further includes prior to the delivery of the ablation energy, receiving, by the processing circuitry a baseline bioelectrical signal from the electrode; and determining the threshold amplitude based on the baseline bioelectrical signal.

Example 7: The method of example 6, wherein estimating the efficacy of the lesion further comprises comparing the first bioelectrical signal to the baseline bioelectrical signal.

Example 8: The method of any of examples 1-7, wherein the bioelectrical signal comprises an intracardiac electrogram (iEGM).

Example 9: The method of example 1-8, wherein the bioelectrical signal is a first bioelectrical signal received at a first time, the method further includes receiving, by the processing circuitry, a second bioelectrical signal from the electrode at second time after the first time; and determining, by the processing circuitry, an amplitude of the second bioelectrical signal, wherein estimating the efficacy of the cardiac lesion further comprises estimating the efficacy of the cardiac lesion based on a comparison of the determined amplitude of the second bioelectrical signal and a second threshold amplitude.

Example 10: The method of examples 1-9, further comprising determining the second threshold based on the baseline signal.

Example 11: The method of examples 1-10, wherein the second time is a predetermined duration subsequent to the first time.

Example 12: The method of examples 1-11, wherein the second time is at least 2 minutes after delivery of ablation energy.

Example 13: The method of example 1, further comprising calculating a lesion durability index based on the determined amplitude of the bioelectrical signal in the frequency band, wherein the lesion durability index comprises a prediction of the efficacy of the cardiac lesion.

Example 14: The method of example 13, further includes performing, by the processing circuitry, a frequency domain analysis on the received bioelectrical signal, wherein the frequency domain analysis comprises dividing the received bioelectrical signal into two or more frequency bands; selecting, by the processing circuitry, a first frequency band of the two or more frequency bands; selecting a second frequency band of the two or more frequency bands; determining, by the processing circuitry, an amplitude of the received bioelectrical signal in the second frequency band; and calculating the lesion durability index based on the amplitude of the bioelectrical signal in the first frequency band and in second frequency band, wherein the lesion durability index comprises a prediction of the efficacy of the cardiac lesion.

Example 15: The method of examples 13 and 14, wherein the second frequency band overlaps the first frequency band.

Example 16: The method of examples 13 and 14, wherein the second frequency band is separate from the first frequency band, and wherein the second frequency band includes higher frequencies than the first frequency band.

Example 17: The method of any one of examples 13-16, wherein a second bioelectrical signal comprises any one or more of: a temperature, an impedance, a pressure, thoracic impedance, cardiac rhythm, a blood chemistry measurement, and an echocardiogram, and wherein the lesion durability index further comprises the second bioelectrical signal.

Example 18: The method of any one of examples 1-17, wherein the electrode is one of a plurality of electrodes; wherein the received bioelectrical signal is bipolar signal, and wherein at least two electrodes of the plurality of electrodes is proximate to the cardiac tissue.

Example 19: The method of any one of examples 1-17, wherein the electrode is one of a plurality of electrodes; wherein the received bioelectrical signal is unipolar signal, and wherein a second electrode of the plurality of electrodes is separate from the first electrode.

Example 20: A medical system configured to perform any of the steps of examples 1 - 19, for example, as described above in relation to FIGS. 1, 2A and 2B.

Example 21: An ablation device configured to perform any of the steps of examples 1 - 20, for example, as described above in relation to FIGS. 1, 2A and 2B.

Example 21: A medical system comprising: an ablation device configured to deliver ablation energy to a target location of cardiac tissue to form a cardiac lesion; sensing circuitry comprising at least one electrode configured to be placed proximate to the target location; and processing circuitry operatively coupled to the sensing circuitry and configured to: receive a bioelectrical signal from the sensing circuitry following conclusion of delivery of the ablation energy; determine an amplitude of the received bioelectrical signal in a frequency band of the received bioelectrical signal; and estimate an efficacy of the cardiac lesion based on a comparison of the determined amplitude of the bioelectrical signal and a threshold amplitude.

An ablation device comprising: ablation generator circuitry configured to deliver ablation energy to a target location of cardiac tissue to form a cardiac lesion; sensing circuitry comprising at least one electrode configured to be placed proximate to the target location; and processing circuitry operatively coupled to the sensing circuitry and configured to: receive a bioelectrical signal from the sensing circuitry following conclusion of delivery of the ablation energy; determine an amplitude of the received bioelectrical signal in a frequency band of the received bioelectrical signal; and estimate an efficacy of the cardiac lesion based on a comparison of the determined amplitude of the bioelectrical signal and a threshold amplitude.

In one or more examples, the functions described above may be implemented in hardware, software, firmware, or any combination thereof. For example, the various components of FIG. 1 may be implemented in hardware, software, firmware, or any combination thereof. If implemented in software, the functions may be stored on a tangible computer-readable storage medium and executed by a processor or hardware-based processing unit..

Instructions may be executed by one or more processors, such as one or more digital signal processors (DSPs), general purpose microprocessors, application specific integrated circuit (ASIC), Field programmable gate array (FPGAs), or other equivalent integrated or discrete logic circuitry. Accordingly, the term “processor,” and “processing circuitry” as used herein, such as may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described

Various examples of the disclosure have been described. These and other examples are within the scope of the following claims.

What is claimed is:

1. A method for evaluating a cardiac lesion formed by an ablation procedure, the method comprising:
receiving, by processing circuitry and following conclusion of delivery of ablation energy, a bioelectrical signal from an electrode proximate to a target location of cardiac tissue for the cardiac lesion;
determining, by the processing circuitry, an amplitude of the received bioelectrical signal in a frequency band of the received bioelectrical signal; and
estimating, by the processing circuitry, an efficacy of the cardiac lesion based on a comparison of the determined amplitude of the bioelectrical signal and a threshold amplitude.

2. The method of claim 1, wherein the ablation energy is pulsed field ablation (PFA).

3. The method of claim 1, wherein the frequency band comprises frequencies less than 30 Hz.

4. The method of claim 1, wherein the frequency band is 0 Hz to 8 Hz.

5. The method of claim 1, wherein subsequent ablation energy is delivered responsive to the estimated efficacy being less than an efficacy threshold.

6. The method of claim 1, wherein the received bioelectrical signal is a first bioelectrical signal, the method further comprising:
prior to the delivery of the ablation energy, receiving, by the processing circuitry a baseline bioelectrical signal from the electrode; and
determining the threshold amplitude based on the baseline bioelectrical signal.

7. The method of claim 6, wherein estimating the efficacy of the lesion further comprises comparing the first bioelectrical signal to the baseline bioelectrical signal.

8. The method of claim 1, wherein the bioelectrical signal comprises an intracardiac electrogram (iEGM).

9. The method of claim 1, wherein the bioelectrical signal is a first bioelectrical signal received at a first time, the method further comprising:
receiving, by the processing circuitry, a second bioelectrical signal from the electrode at a second time after the first time; and
determining, by the processing circuitry, an amplitude of the second bioelectrical signal,
wherein estimating the efficacy of the cardiac lesion further comprises estimating the efficacy of the cardiac lesion based on a comparison of the determined amplitude of the second bioelectrical signal and a second threshold amplitude.

10. The method of claim 9, further comprising determining the second threshold based on the baseline signal.

11. The method of claim 10, wherein the second time is a predetermined duration subsequent to the first time.

12. The method of claim 11, wherein the second time is at least 2 minutes after delivery of ablation energy.

13. The method of claim 1, further comprising calculating a lesion durability index based on the determined amplitude of the bioelectrical signal in the frequency band, wherein the lesion durability index comprises a prediction of the efficacy of the cardiac lesion.

14. The method of claim 13, further comprising:
performing, by the processing circuitry, a frequency domain analysis on the received bioelectrical signal, wherein the frequency domain analysis comprises dividing the received bioelectrical signal into two or more frequency bands;
selecting, by the processing circuitry, a first frequency band of the two or more frequency bands;
selecting a second frequency band of the two or more frequency bands;
determining, by the processing circuitry, an amplitude of the received bioelectrical signal in the second frequency band; and
calculating the lesion durability index based on the amplitude of the bioelectrical signal in the first frequency band and in second frequency band, wherein the lesion durability index comprises a prediction of the efficacy of the cardiac lesion.

15. The method of claim 14, wherein the second frequency band overlaps the first frequency band.

16. The method of claim 14,
wherein the second frequency band is separate from the first frequency band, and
wherein the second frequency band includes higher frequencies than the first frequency band.

17. The method of claim 13,
wherein a second bioelectrical signal comprises any one or more of: a temperature, an impedance, a pressure, thoracic impedance, cardiac rhythm, a blood chemistry measurement, and an echocardiogram, and
wherein the lesion durability index further comprises the second bioelectrical signal.

18. The method of claim 1,
wherein the electrode is one of a plurality of electrodes;
wherein the received bioelectrical signal is bipolar signal, and
wherein at least two electrodes of the plurality of electrodes are proximate to the cardiac tissue.

19. The method of claim 1,
wherein the electrode is a first electrode of a plurality of electrodes;
wherein the received bioelectrical signal is unipolar signal, and
wherein a second electrode of the plurality of electrodes is separate from the first electrode.

20. The method of claim 1, wherein the comparison comprises a ratio of the determined amplitude of the bioelectrical signal and the threshold amplitude.

21. A medical system comprising:
an ablation device configured to deliver ablation energy to a target location of cardiac tissue to form a cardiac lesion;
sensing circuitry comprising at least one electrode configured to be placed proximate to the target location; and
processing circuitry operatively coupled to the sensing circuitry and configured to:
receive a bioelectrical signal from the sensing circuitry following conclusion of delivery of the ablation energy;
determine an amplitude of the received bioelectrical signal in a frequency band of the received bioelectrical signal; and
estimate an efficacy of the cardiac lesion based on a comparison of the determined amplitude of the bioelectrical signal and a threshold amplitude.

22. An ablation device comprising:
ablation generator circuitry configured to deliver ablation energy to a target location of cardiac tissue to form a cardiac lesion;
sensing circuitry comprising at least one electrode configured to be placed proximate to the target location; and
processing circuitry operatively coupled to the sensing circuitry and configured to:
receive a bioelectrical signal from the sensing circuitry following conclusion of delivery of the ablation energy;
determine an amplitude of the received bioelectrical signal in a frequency band of the received bioelectrical signal; and
estimate an efficacy of the cardiac lesion based on a comparison of the determined amplitude of the bioelectrical signal and a threshold amplitude.
